(19) United States
(12) Patent Application Publication
(10) Pub. No.: US 2021/0332107 A1
(43) Pub. Date: Oct. 28, 2021

(54) MODIFIED SERINE PROTEINASE INHIBITORS AND APPLICATIONS THEREOF

(71) Applicant: IONIC BIOMEDICAL INCORPORATED, Ithaca, NY (US)

(72) Inventors: Yadong WANG, Ithaca, NY (US); Hunghao CHU, Moraga, CA (US)

(21) Appl. No.: 17/242,391

(22) Filed: Apr. 28, 2021

Related U.S. Application Data
(60) Provisional application No. 63/016,316, filed on Apr. 28, 2020.

(51) Int. Cl. C07K 14/81; C12N 15/86

(52) U.S. Cl. CPC C07K 14/811; A61K 38/00; C12N 15/86

(57) ABSTRACT

In one aspect, the disclosure relates to modified serine proteinase inhibitors with an enhanced affinity to components of the extracellular matrix, including glycosaminoglycans. The modified serine proteinase inhibitors maintain their original structure and bioactivity while simultaneously exhibiting a higher positive charge density at their glycosaminoglycan-binding surface. Furthermore, the ECM-binding affinity can be tuned by systematically increasing the number of cationic amino acid residues and/or decreasing the number of anionic amino acid residues at the glycosaminoglycan-binding surface. Also disclosed are vectors for producing the modified serine proteinase inhibitors and pharmaceutical conditions incorporating the modified serine proteinase inhibitors.

### CROSS-REFERENCE TO RELATED APPLICATIONS

This application claims the benefit of and priority to co-pending U.S. Provisional Patent Application No. 63/016,316, filed on Apr. 28, 2020, the contents of which are incorporated by reference herein in their entireties.

### BACKGROUND

Serine proteinase inhibitors, or serpins, are members of a large class of proteins found throughout all organism classifications. In humans and other mammals, serpins such as α1-antitrypsin, C1 esterase inhibitor, antithrombin, and plasminogen activator inhibitor are associated with a variety of diseases and medical conditions including, but not limited to, cancers, angiogenesis, disorders of the cardiovascular system, and various ocular disorders. Serpins have further been explored as treatments for wound healing, skin aging, and the like. Serpins have a conserved structure consisting of at least 7 α-helices and 3 β-sheets. For most serpins, serine proteinase inhibitory activity is related to a conformational change from a stressed to a relaxed conformation.

Pigment epithelium-derived factor (PEDF) is a member of the serpin family that does not undergo the characteristic conformational change and exhibits no serine protease inhibitory activity as a result. However, it exhibits structural similarities to active serpins. PEDF is secreted from cells and inhibits angiogenesis as well as influences neuronal differentiation in certain cancers (e.g., retinoblastomas).

The extracellular matrix consists of a variety of biopolymers that offer structural and biochemical support to surrounding cells. The extracellular matrix also attracts a variety of proteins and regulates their activities according to environmental changes. Several important extracellular matrix components are anionic, including glycosaminoglycans such as, for example, hyaluronic acid, heparan sulfate/heparin, chondroitin sulfate, keratan sulfate, and dermatan sulfate. PEDF binds to these glycosaminoglycans; however, this binding can be disrupted by relatively low concentrations of biologically common cations including sodium. What is needed is a modified or engineered serine proteinase inhibitor that retains its original biological activity while exhibiting an increased affinity for negatively-charged glycosaminoglycan components of the extracellular matrix.

Despite advances in the engineering of serine proteinase inhibitors, there is still a need for serpins having enhanced affinity to components of the extracellular matrix (ECM) without compromising the original bioactivity and that are therefore also effective in the treatment of diseases and conditions associated with ECM components. Furthermore, there is a need for serpin derivatives with enhanced half-lives to be therapeutically effective. These needs and other needs are satisfied by the present disclosure.

### SUMMARY

In accordance with the purpose(s) of the present disclosure, as embodied and broadly described herein, the disclosure, in one aspect, relates to modified serine proteinase inhibitors with an enhanced affinity to components of the extracellular matrix, including glycosaminoglycans. The modified serine proteinase inhibitors maintain their original structure and bioactivity while simultaneously exhibiting a higher positive charge density at their glycosaminoglycan-binding surface. Furthermore, the ECM-binding affinity can be tuned by systematically increasing the number of cationic amino acid residues and/or decreasing the number of anionic amino acid residues at the glycosaminoglycan-binding surface. Also disclosed are vectors for producing the modified serine proteinase inhibitors and pharmaceutical conditions incorporating the modified serine proteinase inhibitors.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows the crystal structure of human pigment epithelium-derived factor (PEDF) (PDB ID: 1IMV). The tops images show asymmetrical surface charge distribution: Anionic residues (red) mostly locate around the N-terminus while more cationic residues (blue) are found on the opposite side. In terms of functionality, the N-terminal region contains the bioactive domains for the anti-angiogenic and neurotropic activity shown in, respectively, yellow and green. In contrast, the cationic residues on the opposite side are largely responsible for heparin and hyaluronic acid binding affinities. The crucial cationic residues that involve in binding are highlighted in light blue.

FIG. 2 shows a method for increasing and/or altering surface charge density according to one aspect of the present disclosure. From left to right, the number of blue or positively-charged residues in the protein sequence increases, thus increasing surface charge density. In one aspect, positively-charged residues can be introduced at specific sites, as shown. In an alternative aspect, surface charge density can be increased by random incorporation of positively charged amino acids followed by screening for desired properties.

FIG. 3 shows the net charges of wild type and engineered PEDF under different pH conditions. Between pH 7 and 7.5 (physiological environment), wild type PEDF is negatively-charged. In contrast, engineered PEDF maintains positively-charged, which favors interaction with negatively-charged macromolecules including heparin and hyaluronic acid.

FIG. 4 shows gel electrophoresis of wild type (left panel) and PEDF that has been engineered according to one aspect of the present disclosure. The wild type and engineered PEDF proteins were bound to heparin and eluted with indicated salt solutions. The eluted solutions were concentrated and then analyzed by SDS-PAGE. A higher concentration of NaCl (0.5 M) was required to release engineered PEDF from heparin so that it could enter the gel, while wild type protein required only 0.15 M NaCl. This suggests engineered PEDF has a higher affinity for heparin.

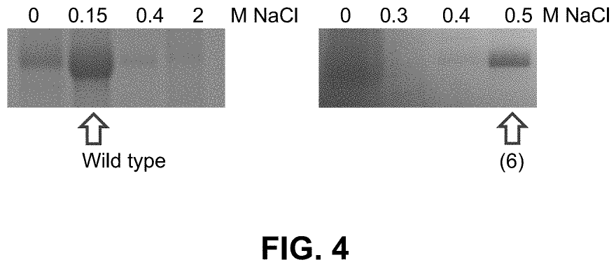

FIG. 5 shows results of a tube-forming assay. In this assay, endothelial cells formed tubes or segments under stimulation from fibroblast growth factor 2 (FGF2); this serves as a model for angiogenesis. Application of both wild type and engineered PEDF decreased the number of segments formed as well as the lengths of the segments; however, engineered PEDF resulted in a slightly greater decrease in number of segments and total segment length, suggesting a slightly higher level of bioactivity for the engineered PEDF protein.

FIG. 6 shows results of a proliferation assay. In this assay, the pro-proliferative effect of vascular endothelial growth factor was counteracted by anti-proliferative PEDF. The similar levels of reduction when both wild type and engineered PEDF were applied revealed that the modifications well preserve this property.

FIG. 7 shows an enzyme-linked immunosorbent assay (ELISA) to measure hyaluronic acid affinity. In this assay, hyaluronic acid was immobilized on a surface and a wild type PEDF as well as two modified PEDF proteins (referred to here as 5.1 and 6), were, in different experiments, bound to the hyaluronic acid. Following binding, amounts of PEDF proteins were detected using an anti-PEDF antibody, with higher values indicating a stronger affinity for hyaluronic acid for the substance being tested.

FIG. 8 shows an ELISA assay to measure collagen-I affinity. The collagen-binding motif of PEDF locates in the N-terminal region and is necessary for the anti-angiogenic activity. In this assay, a 96-well plate was coated with rat collagen-I. Wild type and engineered PEDF (referred to here as 6) were incubated followed by an ELISA assay to determine the amounts of PEDF binding to collagen-I. A similar binding trend suggested that modifications did not affect collagen-I affinity.

FIG. 9 shows a sequence of wild type PEDF protein including distribution of cationic residues. PEDF has 10 α-helices (represented by hA-hJ) and 3 β-sheets (with β-strands being represented by s and wherein, for example, s2A is the second strand of β-sheet A). The residues responsible for hyaluronic acid binding are located in two turns, one between s2A and hE and the other between hF and s3A. According to one aspect of the present disclosure, the engineered PEDF proteins herein should have strengthened hyaluronic acid binding over the motifs from hE to the turn between hF and s3A (shown in yellow).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a modified serine proteinase inhibitor,” “a cationic amino acid residue,” or “an α-helix,” include, but are not limited to, mixtures or combinations of two or more such serine proteinase inhibitors, cationic amino acid residues, or α-helices, and the like.

A “serine proteinase inhibitor” (also “serine protease inhibitor” or “serpin”) is a member of a protein family with a conserved structure that is a common structural class across all organisms and that were first identified for their protease inhibition activity. Serine proteinase inhibitors are involved in various biological processes including, but not limited to, coagulation, inflammation, hormone transport, molecular chaperoning, nutrient storage, chromatin remodeling, and the like.

As used herein, a “cationic amino acid” or “cationic residue” refers to an amino acid incorporated into a polypeptide chain such as, for example, a wild type or modified serine proteinase inhibitor, wherein the side chain of the amino acid takes on a positive charge at neutral pH. In one aspect, the cationic amino acids referred to herein include histidine, arginine, and lysine.

As used herein, an “anionic amino acid” or “anionic residue” refers to an amino acid incorporated into a polypeptide chain such as, for example, a wild type or modified serine proteinase inhibitor, wherein the side chain of the amino acid takes on a negative charge at neutral pH. In one aspect, the anionic amino acids referred to herein include aspartate (aspartic acid) and glutamate (glutamic acid).

An “α-helix” is a secondary structure motif in peptides and proteins. An α-helix is a right-handed helix in which backbone N—H groups hydrogen bond to backbone C═O groups 3-4 amino acids earlier in the protein sequence. α-helices are commonly found in hydrophobic environments such as protein interiors or traversing cell membranes.

A “β-sheet” is a secondary structure motif in proteins and peptides. A β-sheet is made up of at least 2-3 “β-strands,” which are polypeptides from 3-10 amino acid residues in length. The backbone in β-strands is typically extended rather than folded, and β-strands making up the same β-sheet can be found in distant parts of a peptide molecule, but are connected upon protein folding by hydrogen bonds to form a pleated, somewhat twisted sheet.

A “turn” as used herein is a secondary structure motif in proteins and peptides, wherein the backbone reverses direction. A turn is typically marked by two end residues with varying numbers of amino acid residues separating them (i.e., an α-turn with 4 peptide bonds in between, or a β-turn with 3 peptide bonds in between). Some turns may have internal hydrogen bonding.

As used herein, “isoelectric point” (pI) is the pH at which a molecule does not have a net electrical charge. In one aspect, the pH of the human body is about 7.4 and the modified serine proteinase inhibitors disclosed herein have an isoelectric point of greater than 7.4; thus, the modified serine proteinase inhibitors exhibit a net positive charge at pH 7.4. In a further aspect, this net positive charge results in a greater affinity of the modified serine proteinase inhibitors for negatively-charged components of the extracellular matrix.

A “DNA construct” as used herein refers to a segment of linear DNA containing a gene of interest. The gene can be a wild type or naturally-occurring gene or one that has been modified by a method such as, for example, site-directed mutagenesis, and can be a cDNA or can be composed of introns and exons. A DNA construct will typically contain overhangs on each end that correlate to restriction enzyme recognition sites so that the DNA construct can be inserted into a plasmid or other vector containing a multiple cloning site with recognition sites for the same restriction enzyme.

A “vector” as used herein refers to a DNA molecule that carries foreign genetic material into a cell. In one aspect, following transfection into a cell, the vector may be replicated and/or expressed. In another aspect, a vector can be a plasmid, a viral vector, a cosmid, or an artificial chromosome. A typical vector will include an origin of replication (if the vector is intended to reproduce itself), a multiple cloning site that contains restriction sites for different restriction enzymes for ease of insertion of transgenes, and a selection marker such as, for example in bacteria, a gene that confers resistance to an antibiotic. In one aspect, vectors useful herein include plasmids. In an alternative aspect, the vector can be a viral vector such as, for example, a DNA virus. In one aspect, the DNA virus can be an adenovirus or an adeno-associated virus. In some aspects, the vector can be an RNA vector. In one aspect, the RNA vector contains RNA complementary to the DNA disclosed herein. In a further aspect, the vector can be an RNA virus such as, for example, a lentivirus or Sendai virus. In another aspect, the vector can be an mRNA vector.

A “plasmid” is a circular piece of extrachromosomal DNA. Microbial plasmids typically include all the elements required to replicate independently, while mammalian plasmids must be engineered with an origin of replication in order to do so. Genes encoding proteins, including modified or engineered proteins, can be inserted into plasmids, which are then transfected into cells. The cells can then be cultured to produce the proteins encoded by the genes. Non-limiting examples of plasmids useful herein include pWLneo, pSV2cat, pOG44, pXT1, pSG, pSVK3, pBSK, pBSKII, pYES, pYES2, pUC, pUC19, pETDuet-1, p3xFLAG, pBApo, pBI, pcDNA, pCEP, pCI, pCMV, pCTAP, pDEST, pEF, pFLAG, pFN, phCMV, pHet, pIRES, pmRi, pNTAP, prHom, pSecTag, pT-Rex, pTet, pTracer, pTRE, pVAX, pX330, pZeoSV2, derivatives or variants thereof, and combinations thereof.

As used herein, “host cells” are cells used to express a protein such as a modified serine proteinase inhibitor as disclosed herein. In one aspect, the host cells are transfected with a plasmid or other vector containing a DNA construct engineered to express the modified serine proteinase inhibitor. Further in this aspect, the host cells can be cultured under conditions conducive for producing the protein, which is secreted into the culture medium, collected, and purified for further use. In one aspect, the host cells come from a mammal such as, for example, a human. In an alternative aspect, the host cells can be E. coli cells. In another aspect, when the host cells are eukaryotic, the modified serine proteinase inhibitors disclosed herein will exhibit glycosylation patterns and other post-translational modifications similar to native proteins expressed in mammalian tissues.

As used herein, the “extracellular matrix” (ECM) is a network of macromolecules outside the cells. The ECM can include enzymes and glycoproteins as well as structural proteins such as, for example, collagen and elastin. The ECM provides structural support as well as biochemical support to cells and may be involved with cell adhesion, cell communication, and directing cellular differentiation. The ECM can vary in composition and structure in different tissue types. Glycosaminoglycans (GAGs) such as heparan sulfate, chondroitin sulfate, and keratan sulfate found in the ECM participate in the formation of proteoglycans, while polysaccharides such as hyaluronic acid provides the ability of tissues to resist compression due to its binding a large amount of water. In one aspect, both glycosaminoglycans and hyaluronic acid exhibit high levels of negative charges on their surfaces. In a further aspect, the modified serine proteinase inhibitors disclosed herein exhibit a strong affinity for the negatively charged GAGs and hyaluronic acid.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a plasmid refers to an amount that is sufficient to achieve the desired improvement in the property modulated by the formulation component, e.g. achieving uptake by a sufficient number of cells to produce enough of the plasmid-encoded protein for further experimental or therapeutic use. The specific level in terms of wt % in a composition, or of actual numbers of plasmids required as an effective amount will depend upon a variety of factors including the number and type of plasmids used, number and type of host cells, amount of protein desired, and volume of cell culture medium.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Modified Serine Proteinase Inhibitors

In one aspect, disclosed herein are modified serine proteinase inhibitors. In a further aspect, in the modified serine proteinase inhibitors disclosed herein, cationic and anionic amino acid residues are present. In a still further aspect, the sum of the cationic amino acid residues is greater than the sum of the anionic residues in the modified serine proteinase inhibitors.

In a further aspect, achieving a sum of cationic amino acid residues that is greater than the sum of anionic amino acid residues can be accomplished in one of several ways. In one aspect, one or more anionic amino acid residues of the naturally-occurring or wild type serine proteinase inhibitor can be replaced with one or more cationic amino acid residues. In another aspect, one or more anionic amino acid residues of the naturally-occurring or wild type serine proteinase inhibitor can be removed. In still another aspect, one or more cationic acid residues can be added to the naturally-occurring or wild type serine proteinase inhibitor.

In another aspect, each of these techniques can be used alone to modify the amino acid residues, or the techniques can be used in any combination to modify the amino acid residues. In one aspect, the cationic amino acid residue can be lysine, arginine, histidine, or any combination thereof. In another aspect, the anionic amino acid residue can be aspartic acid/aspartate, glutamic acid/glutamate, or a combination thereof.

In any of these aspects, addition, removal, or replacement of amino acid residues can be accomplished by site-directed mutagenesis of the corresponding cDNA. In one aspect, when removal of an amino acid is desired, the DNA codon representing that amino acid can be removed from the cDNA sequence by any technique known in the art. In another aspect, when addition of an amino acid is desired, a DNA codon representing that amino acid can be inserted into the cDNA sequence by any technique known in the art. In still another aspect, when it is desired to substitute one amino acid for another, one or more bases of the cDNA codon for a first amino acid can be changed to generate a codon for a second amino acid. Thus, for example, if a cDNA sequence includes a GAA codon for glutamate, and it is desired to replace the glutamate with lysine, the first DNA base can be mutated from G to A via site directed mutagenesis or another technique, so the codon changed from GAA to AAA, which is associated with lysine.

In one aspect, position 601 in SEQ ID NOS. 1 and 2 is adenine. In another aspect, positions 601-604 in SEQ ID NOS. 1 and 2 is AAGA. In another aspect, positions 601-604 in SEQ ID NOS. 1 and 2 is AAGA and positions 577-579 are AAG. In another aspect, positions 601-604 in SEQ ID NOS. 1 and 2 is AAGA, positions 577-579 are AAG, and positions 555-557 are AAG.

In one aspect, the sum of lysine and arginine residues is greater than the sum of the aspartate and glutamate residues in the modified serine proteinase inhibitor. In one aspect, the sum of the lysine and arginine residues is from 46 to 55, or is 46, 47, 48, 49, 50, 51, 52, 53, 54, or 55, or a combination of any of the foregoing values, or a range encompassing any of the foregoing values.

### Structural Considerations

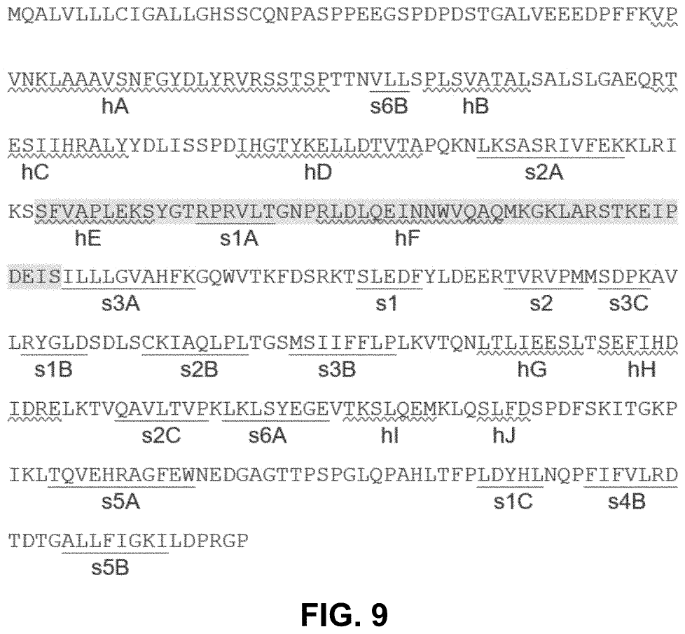

In one aspect, naturally occurring as well as engineered serine proteinase inhibitors typically have 7, 8, 9, or 10 α-helices (represented herein by hA through hJ) and 3 β-sheets, with each β-sheet being made up of two or more β-strands (represented by s1A, s4B, and so on, where s1A is the first strand of β-sheet A, etc.), which can be from adjacent parts of the protein sequence or which can be more distant from one another in the protein sequence and come together into a β-sheet structure through protein folding mechanisms. In a still further aspect, turns can be found between various structural elements in the protein sequence. In any of these aspects, the secondary structure of serine proteinase inhibitors (α-helices, β-sheets, β-strands, and turns) is generally conserved among members of the serine proteinase inhibitor family. As seen in FIG. 9, in one aspect, hE and hF represent the fifth and sixth α-helices, respectively, in the conserved family structure, while s2A and s3A represent the second and third β-strands, respectively, of β-sheet A in the conserved family structure.

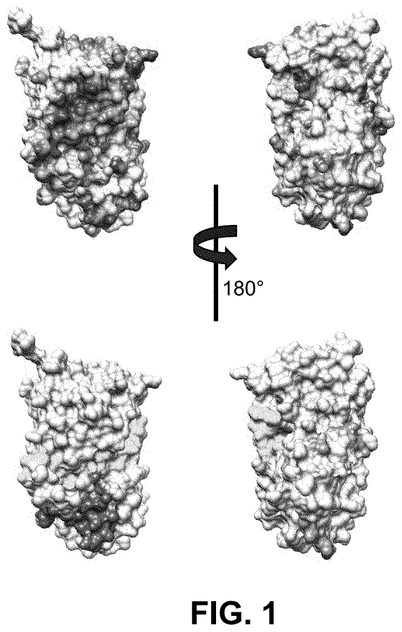

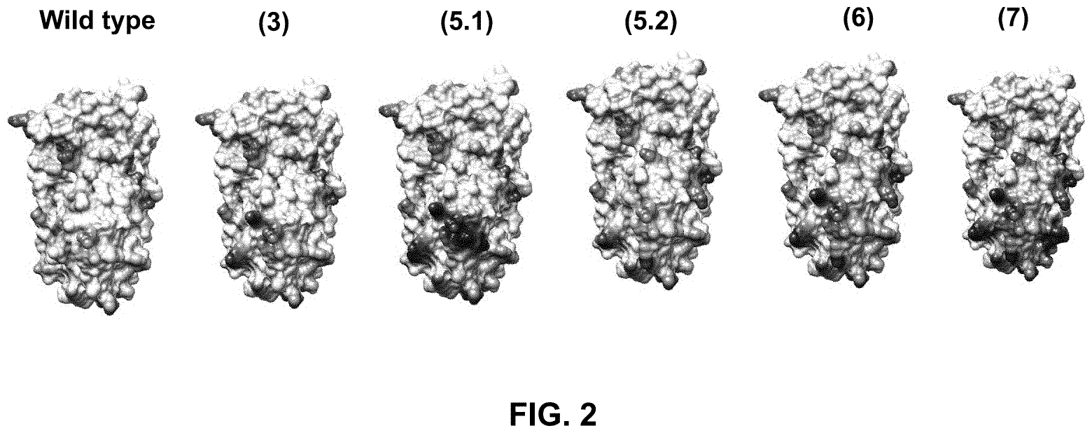

In one aspect, binding of components of the extracellular matrix to serine proteinase inhibitors such as, for example, PEDF, can occur at a turn between s2A and hE as well as a turn between hF and s3A in the naturally-occurring or wild type serine proteinase inhibitors. Thus, in a further aspect, addition, removal, or substitution of amino acid residues to achieve the desired ratio of cationic to anionic amino acid residues can be performed in the turn regions between s2A and hE, between hF and s3A, or in another location. In one aspect, when the serine proteinase inhibitor is folded into a tertiary structure or three-dimensional configuration, these turn regions appear on one side or face of the tertiary structure (see FIG. 1) and thus, when modifications are made to increase the number of cationic residues in these regions, more cationic residues appear on that side or face (see FIG. 1). Thus, in this aspect, positive charges are concentrated on one side or face of the serine proteinase inhibitor for aid in binding to components of the negatively-charged extracellular matrix. In another aspect, increasing the number of cationic residues increases surface charge density for the folded protein (FIG. 2). In another aspect, other serpins may bind to targets at other locations along their sequences. In one aspect, for serpins that do not typically bind their targets in the turn regions between s2A and hE and/or between hF and s3A, binding to glycosaminoglycans can be engineered by altering the protein sequences to incorporate additional cationic residues in the disclosed turn regions. In a still further aspect, glycosaminoglycan binding may also occur at hD, the region bet ween hH and s2C, or the like. In any of these aspects, amino acid substitution is performed outside of the active site of the serine proteinase inhibitor. Further in this aspect, the active sites retain their conformation and catalytic residues and, thus, enzymatic or other biological activity is not affected by the disclosed substitutions.

### Chemical Properties

In one aspect, increasing the ratio of cationic amino acid residues relative to anionic amino acid residues shifts the isoelectric point of the modified serine proteinase inhibitor disclosed herein. In one aspect, the modified serine proteinase inhibitor has an isoelectric point (pI) that is from about 10% to about 90% greater than the naturally-occurring serine proteinase inhibitor, or is about 10, 15, 20, 25, 30, 35, 40, 45, 50, 55, 60, 65, 70, 75, 80, 85, or about 90% greater than the naturally-occurring serine proteinase inhibitor, or a combination of any of the foregoing values, or a range encompassing any of the foregoing values. In one aspect, the pI is at least 7.4, or about 7.4, about 7.6, about 7.8, about 8.0, about 8.2, about 8.4, about 8.6, about 8.8, about 9.0, about 9.2, about 9.4, about 9.6, about 9.8, or about 10, or a combination of any of the foregoing values, or a range encompassing any of the foregoing values. In another aspect, the pI continues to increase as additional cationic residues are added to the modified serine proteinase inhibitor.

Serine Proteinase Inhibitors

In one aspect, the modified serine proteinase inhibitor disclosed herein can be selected from α1-antitrypsin, C1 esterase inhibitor, antithrombin, plasminogen activator inhibitor, pigment epithelium-derived factor, antitrypsin-related protein, α1-antichymotrypsin, kallistatin, protein C inhibitor, transcortin, thyroxin-binding globulin, antiangiotensinogen, centerin, protein Z-related protease inhibitor, vaspin, monocyte neutrophil elastase inhibitor, squamous cell carcinoma antigen, maspin, megsin, bomapin, yukopin, hurpin/headpin, heparin cofactor II, glia derived nexin, α2-antiplasmin, complement 1 inhibitor, 47 kDa heat shock protein, neuroserpin, pancpin, or any combination thereof.

In another aspect, the modified serine proteinase inhibitor can be pigment epithelium-derived factor (PEDF) and the sum of the cationic amino acid residues between hF and s3A is greater than the sum of anionic amino acid residues between hF and s3A. In still another aspect, the modified serine proteinase inhibitor can be pigment epithelium-derived factor (PEDF) and the sum of the cationic amino acid residues between hE and s2A is greater than the sum of anionic amino acid residues between hE and s2A.

In one aspect, the modified serine proteinase inhibitor differs by at least one amino acid from the protein of SEQ ID NO. 3 (i.e., wild type or naturally-occurring protein). In another aspect, the modified serine proteinase inhibitor can be generated by varying at least one codon in cDNA sequence SEQ ID NO. 1 or 2. In another aspect, the cDNA encoding the modified serine proteinase inhibitor can have SEQ ID NO. 4, 6, 8, 10, or 12, or at least 70%, 75%, 80%, 85%, 90%, 95%, or 99% homology thereto, or a combination of any of the foregoing values, or a range encompassing any of the foregoing values. In still another aspect, the modified serine proteinase inhibitor can have SEQ ID NO. 5, 7, 9, 11, or 13, or at least 70%, 75%, 80%, 85%, 90%, 95%, or 99% homology thereto, or a combination of any of the foregoing values, or a range encompassing any of the foregoing values.

### DNA Constructs, Vectors, and Biological Devices

### DNA Constructs

DNA constructs are provided herein for the production of biological devices useful as for the production of modified serine proteinase inhibitors. It is understood that one way to define the variants and derivatives of the genetic components and DNA constructs described herein is in terms of homology/identity to specific known sequences. Those of skill in the art readily understand how to determine the homology of two nucleic acids. For example, the homology can be calculated after aligning two sequences so that the homology is at its highest level. Another way of calculating homology can be performed according to published algorithms (see Zuker, M., Science, 244:48-52, 1989; Jaeger et al., Proc. Natl. Acad. Sci. USA, 86:7706-7710, 1989; Jaeger et al., Methods Enzyol. 183:281-306, 1989, which are herein incorporated by reference for at least material related to nucleic acid alignment).

As used herein, “conservative” mutations are mutations that result in an amino acid change in the protein produced from a sequence of DNA. When a conservative mutation occurs, the new amino acid has similar properties as the wild type amino acid and generally does not drastically change the function or folding of the protein (e.g., switching isoleucine for valine is a conservative mutation since both are small, branched, hydrophobic amino acids). “Silent mutations,” meanwhile, change the nucleic acid sequence of a gene encoding a protein but do not change the amino acid sequence of the protein.

It is understood that the description of mutations and homology can be combined together in any combination, such as embodiments that have at least 70%, 75%, 80%, 85%, 90%, 95%, or 99% homology to a particular sequence wherein the variants are conservative or silent mutations. It is understood that any of the sequences described herein can be a variant or derivative having the homology values listed above.

In one aspect, a database such as, for example, GenBank, can be used to determine the sequences of genes and/or regulatory regions of interest, the species from which these elements originate, and related homologous sequences.

In one aspect, the nucleic acids described herein (e.g., genes that express wild type and/or modified serine proteinase inhibitors) used in the DNA constructs described herein can be amplified using polymerase chain reaction (PCR) prior to being ligated into a plasmid or other vector. Typically, PCR-amplification techniques make use of primers, or short, chemically-synthesized oligonucleotides are complementary to regions on each respective strand flanking the DNA or nucleotide sequence to be amplified. A person having ordinary skill in the art will be able to design or choose primers based on the desired experimental conditions. In general, primers should be designed to provide for both efficient and faithful replication of the target nucleic acids. Two primers are required for the amplification of each gene, one for the sense strand (that is, the strand containing the gene of interest) and one for the antisense strand (that is, the strand complementary to the gene of interest). Pairs of primers should have similar melting temperatures that are close to the PCR reaction's annealing temperature. In order to facilitate the PCR reaction, the following features should be avoided in primers: mononucleotide repeats, complementarity with other primers in the mixture, self-complementarity, and internal hairpins and/or loops. Methods of primer design are known in the art; additionally, computer programs exist that can assist the skilled practitioner with primer design. Primers can optionally incorporate restriction enzyme recognition sites at their 5′ ends to assist in later ligation into plasmids or other vectors. In some aspects, the DNA can be transcribed into RNA and incorporated into an RNA vector such as, for example, an RNA virus or an mRNA vector.

PCR can be carried out using purified DNA, unpurified DNA that is integrated into a vector, or unpurified genomic DNA. The process for amplifying target DNA using PCR consists of introducing an excess of two primers having the characteristics described above to a mixture containing the sequence to be amplified, followed by a series of thermal cycles in the presence of a heat-tolerant or thermophilic DNA polymerase, such as, for example, any of Taq, Pfu, Pwo, Tfl, rTth, Tli, or Tma polymerases. A PCR “cycle” involves denaturation of the DNA through heating, followed by annealing of the primers to the target DNA, followed by extension of the primers using the thermophilic DNA polymerase and a supply of deoxynucleotide triphosphates (i.e., dCTP, dATP, dGTP, and TTP), along with buffers, salts, and other reagents as needed. In one aspect, the DNA segments created by primer extension during the PCR process can serve as templates for additional PCR cycles. Many PCR cycles can be performed to generate a large concentration of target DNA or gene. PCR can optionally be performed in a device or machine with programmable temperature cycles for denaturation, annealing, and extension steps. Further, PCR can be performed on multiple genes simultaneously in the same reaction vessel or microcentrifuge tube since the primers chosen will be specific to selected genes. PCR products can be purified by techniques known in the art such as, for example, gel electrophoresis followed by extraction from the gel using commercial kits and reagents.

In one aspect, the nucleic acids described herein used in the DNA constructs can be synthesized by stepwise addition of nucleotides (a.k.a. gene synthesis) to produce a single-stranded cDNA, which then serves as the template for the complimentary strand for site-directed mutagenesis.

In a further aspect, the plasmid can include an origin of replication, allowing it to use the host cell's replication machinery to create copies of itself.

As used herein, “operably linked” refers to the association of nucleic acid sequences on a single nucleic acid fragment so that the function of one affects the function of another. For example, if sequences for multiple genes are inserted into a single plasmid, their expression may be operably linked. Alternatively, a promoter is said to be operably linked with a coding sequence when it is capable of affecting the expression of that coding sequence.

As used herein, “expression” refers to transcription and/or accumulation of an mRNA derived from a gene or DNA fragment. Expression may also be used to refer to translation of mRNA into a peptide, polypeptide, or protein.

In one aspect, the DNA constructs disclosed herein incorporate a gene that expresses a modified serine proteinase inhibitor. In a further aspect, the gene that expresses a modified serine proteinase inhibitor has SEQ ID NO. 4, 6, 8, 10, 12, or at least 70% homology thereto, at least 75% homology thereto, at least 80% homology thereto, at least 85% homology thereto, at least 90% homology thereto, at least 95% homology thereto, or at least 99% homology thereto.

In one aspect, disclosed herein is a DNA construct having or including SEQ ID NO. 4, 6, 8, 10, 12, or at least 70%, 75, 80, 85, 90, 95, or 99% homology thereto, or a combination of any of the foregoing values, or a range encompassing any of the foregoing values.

In one aspect, the construct includes a regulatory sequence. In a further aspect, the regulatory sequence is already incorporated into a vector such as, for example, a plasmid, prior to genetic manipulation of the vector. In another aspect, the regulatory sequence can be incorporated into the vector through the use of restriction enzymes or any other technique known in the art.

In one aspect, the regulatory sequence is a promoter. The term “promoter” refers to a DNA sequence capable of controlling the expression of a coding sequence. In another aspect, the coding sequence to be controlled is located 3′ to the promoter. In another aspect, the promoter is derived from a native gene. In an alternative aspect, the promoter is composed of multiple elements derived from different genes and/or promoters. A promoter can be assembled from elements found in nature, from artificial and/or synthetic elements, or from a combination thereof. It is understood by those skilled in the art that different promoters can direct the expression of a gene in different tissues or cell types, at different stages of development, in response to different environmental or physiological conditions, and/or in different species. In one aspect, the promoter functions as a switch to activate the expression of a gene.

In one aspect, the promoter is “constitutive.” A constitutive promoter is a promoter that causes a gene to be expressed in most cell types at most times. In another aspect, the promoter is “regulated.” A regulated promoter is a promoter that becomes active in response to a specific stimulus. A promoter may be regulated chemically, such as, for example, in response to the presence or absence of a particular metabolite (e.g., lactose or tryptophan), a metal ion, a molecule secreted by a pathogen, or the like. A promoter also may be regulated physically, such as, for example, in response to heat, cold, water stress, salt stress, oxygen concentration, illumination, wounding, or the like.

In another aspect, the regulatory sequence is a terminator or stop sequence. As used herein, a terminator is a sequence of DNA that marks the end of a gene or operon to be transcribed. In a further aspect, the terminator is an intrinsic terminator or a Rho-dependent transcription terminator. As used herein, an intrinsic terminator is a sequence wherein a hairpin structure can form in the nascent transcript that disrupts the mRNA/DNA/RNA polymerase complex. As used herein, a Rho-dependent transcription terminator requires a Rho factor protein complex to disrupt the mRNA/DNA/RNA polymerase complex. In one aspect, the terminator is a T7 terminator. In an alternative aspect, the terminator is a CYC1 terminator obtained from or native to the pYES2 plasmid.

In a further aspect, the regulatory sequence includes both a promoter and a terminator or stop sequence. In a still further aspect, the regulatory sequence can include multiple promoters or terminators. Other regulatory elements, such as enhancers, are also contemplated. Enhancers may be located from about 1 to about 2000 nucleotides in the 5′ direction from the start codon of the DNA to be transcribed, or may be located 3′ to the DNA to be transcribed. Enhancers may be “cis-acting,” that is, located on the same molecule of DNA as the gene whose expression they affect.

In one aspect, when the vector is a plasmid, the plasmid can also contain a multiple cloning site or polylinker. In a further aspect, the polylinker contains recognition sites for multiple restriction enzymes. The polylinker can contain up to 2, 3, 4, 5, 6, 7, 8, 9, 10, 11, 12, 13, 14, 15, 16, 17, 18, 19, 20, or more than 20 recognition sites for restriction enzymes. Further, restriction sites may be added, disabled, or removed as required, using techniques known in the art. In one aspect, the plasmid contains restriction sites for any known restriction enzyme such as, for example, HindIII, KpnI, SacI, BamHI, BstXI, EcoRI, BasBI, NotI, XhoI, SphI, XbaI, ApaI, SalI, ClaI, EcoRV, PstI, SmaI, XmaI, SpeI, EagI, SacII, or any combination thereof. In a further aspect, the plasmid contains more than one recognition site for the same restriction enzyme.

In one aspect, the restriction enzyme can cleave DNA at a palindromic or an asymmetrical restriction site. In a further aspect, the restriction enzyme cleaves DNA to leave blunt ends; in an alternative aspect, the restriction enzyme cleaves DNA to leave “sticky” or overhanging ends. In another aspect, the enzyme can cleave DNA to a distance of from 20 bases to over 1000 bases away from the restriction site. A variety of restriction enzymes are commercially available and their recognition sequences, as well as instructions for use (e.g., amount of DNA needed, precise volumes or reagents, purification techniques, as well as information about salt concentration, pH, optimum temperature, incubation time, and the like) are provided by enzyme manufacturers.

In one aspect, a plasmid with a polylinker containing one or more restriction sites can be digested with one restriction enzyme and a nucleotide sequence of interest can be ligated into the plasmid using a commercially-available DNA ligase enzyme. Several such enzymes are available, often as kits containing all reagents and instructions required for use. In another aspect, a plasmid with a polylinker containing two or more restriction sites can be simultaneously digested with two restriction enzymes and a nucleotide sequence of interest can be ligated into the plasmid using a DNA ligase enzyme. Using two restriction enzymes provides an asymmetric cut in the DNA, allowing for insertion of a nucleotide sequence of interest in a particular direction and/or on a particular strand of the double-stranded plasmid. Since RNA synthesis from a DNA template proceeds from 5′ to 3′, usually starting just after a promoter, the order and direction of elements inserted into a plasmid can be especially important. If a plasmid is to be simultaneously digested with multiple restriction enzymes, these enzymes must be compatible in terms of buffer, salt concentration, and other incubation parameters.

In some aspects, prior to ligation using a ligase enzyme, a plasmid that has been digested with a restriction enzyme is treated with an alkaline phosphatase enzyme to remove 5′ terminal phosphate groups. This prevents self-ligation of the plasmid and thus facilitates ligation of heterologous nucleotide fragments into the plasmid.

The DNA construct described herein can be part of a vector. In a further aspect, the vector is a plasmid, a phagemid, a cosmid, a yeast artificial chromosome, a bacterial artificial chromosome, a virus, a phage, or a transposon. In general, plasmid vectors containing replicon and control sequences that are derived from species compatible with the host cell are used in connection with the hosts. The vector ordinarily carries a replication site as well as marking sequences that are capable of performing phenotypic selection in transformed cells. Plasmid vectors are well known and are commercially available. Such vectors include, but are not limited to, pWLneo, pSV2cat, pOG44, pXT1, pSG, pSVK3, pBSK, pBSKII, pYES, pYES2, pUC, pUC19, pETDuet-1, p3xFLAG, pBApo, pBI, pcDNA, pCEP, pCI, pCMV, pCTAP, pDEST, pEF, pFLAG, pFN, phCMV, pHet, pIRES, pmRi, pNTAP, prHom, pSecTag, pT-Rex, pTet, pTracer, pTRE, pVAX, pX330, and pZeoSV2 vectors.

Plasmids are double-stranded, autonomously-replicating, genetic elements that are not integrated into host cell chromosomes. Further, these genetic elements are usually not part of the host cell's central metabolism. In bacteria, plasmids may range from 1 kilobase (kb) to over 200 kb. Plasmids can be engineered to encode a number of useful traits including the production of secondary metabolites, antibiotic resistance, the production of useful proteins, degradation of complex molecules and/or environmental toxins, and others. Plasmids have been the subject of much research in the field of genetic engineering, as plasmids are convenient expression vectors for foreign DNA in, for example, microorganisms. Plasmids generally contain regulatory elements such as promoters and terminators and also usually have independent replication origins. Ideally, plasmids will be present in multiple copies per host cell and will contain selectable markers (such as genes for antibiotic resistance) to allow the skilled artisan to select host cells that have been successfully transfected with the plasmids (for example, by growing the host cells in a medium containing the antibiotic).

Vectors capable of high levels of expression of recombinant genes and proteins are well known in the art. Vectors useful for the transformation of a variety of host cells are common and commercially available and include. The skilled practitioner will be able to choose a plasmid based on such factors as a) the amount of nucleic acid (i.e., number of genes and other elements) to be inserted, b) the host organism, c) culture conditions for the host organism, and other related factors.

In one aspect, the vector encodes a selective marker. In a further aspect, the selective marker is a gene that confers resistance to an antibiotic. In certain aspects, during fermentation of host cells transformed with the vector, the cells are contacted with the antibiotic. For example, the antibiotic may be included in the culture medium. Cells that have not been successfully transformed cannot survive in the presence of the antibiotic; only cells containing the vector which confers antibiotic resistance can survive. Optimally, only cells containing the vector to be expressed will be cultured, as this will result in the highest production efficiency of the desired gene products (e.g., peptides). Cells that do not contain the vector would otherwise compete with transformed cells for resources. In one aspect, the antibiotic is tetracycline, neomycin, kanamycin, ampicillin, hygromycin, chloramphenicol, amphotericin B, bacitracin, carbapenam, cephalosporin, ethambutol, fluoroquinolones, isonizid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

In another aspect, disclosed herein is a vector that includes the disclosed DNA construct. In one aspect, the vector is a plasmid. In another aspect, the plasmid is selected from pWLneo, pSV2cat, pOG44, pXT1, pSG, pSVK3, pBSK, pBSKII, pYES, pYES2, pUC, pUC19, pETDuet-1, p3xFLAG, pBApo, pBI, pcDNA, pCEP, pCI, pCMV, pCTAP, pDEST, pEF, pFLAG, pFN, phCMV, pHet, pIRES, pmRi, pNTAP, prHom, pSecTag, pT-Rex, pTet, pTracer, pTRE, pVAX, pX330, pZeoSV2, a derivative or variant thereof, or a combination thereof. In an alternative aspect, the vector can be a DNA virus such as, for example, an adenovirus or adeno-associated virus. In still another aspect, the vector can be an RNA vector such as, for example, a lentivirus, Sendai virus, or an mRNA vector. Further in this aspect, the RNA in the vector can be complementary to the DNA sequences disclosed herein.

### Biological Devices

In one aspect, a “biological device” is formed when a microbial cell is transfected with the DNA construct described herein. The biological devices are generally composed of microbial host cells, where the host cells are transformed with a DNA construct described herein.

In one aspect, the DNA construct is carried by the expression vector into the cell and is separate from the host cell's genome. In another aspect, the DNA construct is incorporated into the host cell's genome. In still another aspect, incorporation of the DNA construct into the host cell enables the host cell to produce modified serine proteinase inhibitors. “Heterologous” genes and proteins are genes and proteins that have been experimentally inserted into a cell that are not normally expressed by the cell. A heterologous gene may be cloned or derived from a different cell type or species than the recipient cell or organism. Heterologous genes may be introduced into cells by transduction or transformation.

An “isolated” nucleic acid is one that has been separated from other nucleic acid molecules and/or cellular material (peptides, proteins, lipids, saccharides, and the like) normally present in the natural source of the nucleic acid. An “isolated” nucleic acid may optionally be free of the flanking sequences found on either side of the nucleic acid as it naturally occurs. An isolated nucleic acid can be naturally occurring, can be chemically synthesized, or can be a cDNA molecule (i.e., is synthesized from an mRNA template using reverse transcriptase and DNA polymerase enzymes).

“Transformation” or “transfection” as used herein refers to a process for introducing heterologous DNA into a host cell. Transformation can occur under natural conditions or may be induced using various methods known in the art. Many methods for transformation are known in the art and the skilled practitioner will know how to choose the best transformation method based on the type of cells being transformed. Methods for transformation include, for example, viral infection, electroporation, lipofection, chemical transformation, and particle bombardment. Cells may be stably transformed (i.e., the heterologous DNA is capable of replicating as an autonomous plasmid or as part of the host chromosome) or may be transiently transformed (i.e., the heterologous DNA is expressed only for a limited period of time).

“Competent cells” refers to microbial cells capable of taking up heterologous DNA. Competent cells can be purchased from a commercial source, or cells can be made competent using procedures known in the art.

The host cells as referred to herein include their progeny, which are any and all subsequent generations formed by cell division. It is understood that not all progeny may be identical due to deliberate or inadvertent mutations. A host cell may be “transfected” or “transformed,” which refers to a process by which exogenous nucleic acid is transferred or introduced into the host cell.

A transformed cell includes the primary subject cell and its progeny. The host cells can be naturally-occurring cells or “recombinant” cells. Recombinant cells are distinguishable from naturally-occurring cells in that naturally-occurring cells do not contain heterologous DNA introduced through molecular cloning procedures. In one aspect, the host cell is a prokaryotic cell such as, for example, Escherichia coli. In other aspects, the host cell is a eukaryotic cell such as, for example, the yeast Saccharomyces cerevisiae, a cell from a human or another mammal, or an insect cell. Host cells transformed with the DNA construct described herein are referred to as “biological devices.”

The DNA construct is first delivered into the host cell. In one aspect, the host cells are naturally competent (i.e., able to take up exogenous DNA from the surrounding environment). In another aspect, cells must be treated to induce artificial competence. This delivery may be accomplished in vitro, using well-developed laboratory procedures for transforming cell lines. Transformation of bacterial cell lines can be achieved using a variety of techniques. One method involves calcium chloride. The exposure to the calcium ions renders the cells able to take up the DNA construct. Another method is electroporation. In this technique, a high-voltage electric field is applied briefly to cells, producing transient holes in the membranes of the cells through which the vector containing the DNA construct enters. Another method involves exposing intact yeast cells to alkali cations such as, for example, lithium. In one aspect, this method includes exposing yeast to lithium acetate, polyethylene glycol, and single-stranded DNA such as, for example, salmon sperm DNA. Without wishing to be bound by theory, the single-stranded DNA is thought to bind to the cell wall of the yeast, thereby blocking plasmids from binding. The plasmids are then free to enter the yeast cell. Enzymatic and/or electromagnetic techniques can also be used alone, or in combination with other methods, to transform microbial cells. Exemplary procedures for transforming yeast and bacteria with specific DNA constructs are provided in the Examples. In certain aspects, two or more types of DNA can be incorporated into the host cells. Thus, different metabolites can be produced from the same host cells at enhanced rates.

In still another aspect, disclosed herein is a biological device including host cells transformed with the DNA construct or vector disclosed herein. In one aspect, the host cells are mammalian cells such as, for example, human embryonic kidney cells, Chinese hamster ovary cells, or another mammalian cell line. In an alternative aspect, the host cells can be bacterial cells such as, for example, E. coli, or can be yeast cells such as, for example, Saccharomyces cerevisiae, or can be insect cells. In a further aspect, when the host cells are mammalian cells, the modified serine proteinase inhibitor can be produced having post-translational modifications such as glycosylation that would be found in the naturally-occurring or wild type protein produced by the original source organism. In some aspects, the post-translational modifications are not needed and the modified serine proteinase inhibitor can be produced in greater amounts and with more efficiency by a bacterial or fungal host.

### Production of Modified Serine Proteinase Inhibitors

The biological devices described herein are useful in the production of modified serine proteinase inhibitors as disclosed herein. Once the DNA construct has been incorporated into the host cell, the cells are cultured such that the cells multiply. A satisfactory microbiological culture contains available sources of hydrogen donors and acceptors, carbon, nitrogen, sulfur, phosphorus, inorganic salts, and, in certain cases, vitamins or other growth-promoting substances. For example, the addition of peptone provides a readily-available source of nitrogen and carbon. Furthermore, the use of different types of media results in different growth rates and different stationary phase densities; stationary phase is where secondary metabolite production occurs most frequently. A rich media results in a short doubling time and higher cell density at stationary phase. Minimal media results in slow growth and low final cell densities. Efficient agitation and aeration increase final cell densities.

In one aspect, host cells can be cultured or fermented by any method known in the art. The skilled practitioner will be able to select a culture medium based on the species and/or strain of host cell selected. In certain aspects, the culture medium will contain a carbon source. A variety of carbon sources are contemplated, including, but not limited to: monosaccharides such as glucose and fructose, disaccharides such as lactose or sucrose, oligosaccharides, polysaccharides such as starch, or mixtures thereof. In one aspect, the biological devices described herein are cultured with a medium composed of raffinose, galactose, or a combination thereof. Unpurified mixtures extracted from feedstocks are also contemplated and include molasses, barley malt, and related compounds and compositions. Other glycolytic and tricarboxylic acid cycle intermediates are also contemplated as carbon sources, as are one-carbon substrates such as carbon dioxide and/or methanol in the cases of compatible organisms. The carbon source utilized is limited only by the particular organism being cultured.

Culturing or fermenting of host cells can be accomplished by any technique known in the art. In one aspect, batch fermentation can be conducted. In batch fermentation, the composition of the culture medium is set at the beginning and the system is closed to future alterations. In some aspects, a limited form of batch fermentation may be carried out, wherein factors such as oxygen concentration and pH are manipulated, but additional carbon is not added. Continuous fermentation methods are also contemplated. In continuous fermentation, equal amounts of a defined medium are continuously added to and removed from a bioreactor. In other aspects, microbial host cells are immobilized on a substrate. Fermentation may be carried out on any scale and may include methods in which literal “fermentation” is carried out as well as other culture methods that are non-fermentative.

In one aspect, the method involves growing the biological devices described herein for a sufficient time to produce proteins encoded by the disclosed genes. The ordinary artisan will be able to choose a culture medium and optimum culture conditions based on the biological identity of the host cells.

In a further aspect, the proteins produced by the biological devices are secreted by the host cells into the culture medium, where they can be collected, separated from the microbial cells (lysed or intact), and/or purified through any technique known in the art such as, for example, precipitation, centrifugation, filtration, and the like. In one aspect, the proteins are passed over an affinity column packed with immobilized hyaluronic acid, rinsed, and eluted with increasing concentrations of sodium chloride. Further in this aspect, following elution, the proteins can be desalted and stored for further use.

In yet another aspect, disclosed herein is a modified serine proteinase inhibitor produced by culturing the biological device disclosed herein. In one aspect, the modified serine proteinase inhibitor is a secretory protein. Further in this aspect, the biological device can be cultured for a sufficient period to produce the modified serine proteinase inhibitor, the culture medium can be collected and concentrated, and the modified serine proteinase inhibitor can be purified from the culture medium. In one aspect, the culture medium containing secreted modified serine proteinase inhibitor can be passed over a column containing immobilized heparin. Further in this aspect, the modified serine proteinase inhibitor binds to heparin while other components of the culture medium pass through the column. In a still further aspect, the modified serine proteinase inhibitor can then be released from the column by flowing increasingly concentrated solutions of sodium chloride through the column to disrupt the ionic and electrostatic interactions binding the modified serine proteinase inhibitor to the immobilized heparin. In one aspect, the concentration of sodium chloride necessary to elute the modified serine proteinase inhibitor is from about 0.15 M to about 0.5 M, or is about 0.15, 0.2, 0.25, 0.3, 0.35, 0.4, 0.45, or about 0.5 M, or a combination of any of the foregoing values, or a range encompassing any of the foregoing values. In a still further aspect, the modified serine proteinase inhibitor can be desalted by any method known in the art (e.g., a spin column filter or another method) and stored for later use.

### Pharmaceutical Compositions

In one aspect, disclosed herein are pharmaceutical compositions for treating a condition relating to increased vascular permeability, a condition relating to increased angiogenesis, an ocular disease, a condition associated with reduced bone mass, skin aging, a cancer, a wound, or a condition associated with inflammation in a subject.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, intravitreal, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ocular disease, a disorder associated with increased angiogenesis, and/or a wound. The effect can be therapeutic in terms of a partial or complete cure of a disease, condition, symptom or adverse effect attributed to the disease, disorder, or condition. The term “treatment” as used herein can include any treatment of ocular diseases, diseases associated with increased angiogenesis, and/or wounds in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed modified serine proteinase inhibitor and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed modified serine proteinase inhibitor or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed modified serine proteinase inhibitor and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed modified serine proteinase inhibitor and/or pharmaceutical composition, by changing the disclosed modified serine proteinase inhibitor and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When modified serine proteinase inhibitors of the present disclosure contain relatively acidic functionalities, base addition salts can be obtained by contacting the neutral form of such modified serine proteinase inhibitors with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When modified serine proteinase inhibitors of the present disclosure contain relatively basic functionalities, acid addition salts can be obtained by contacting the neutral form of such modified serine proteinase inhibitors with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of modified serine proteinase inhibitors of the present disclosure which hydrolyze in vivo and include those that break down readily in the human body to leave the parent protein or a salt thereof. Examples of pharmaceutically acceptable, non-toxic esters of the present disclosure include C1-to-C6 alkyl esters and C5-to-C7 cycloalkyl esters, although C1-to-C4 alkyl esters are preferred. Esters of disclosed modified serine proteinase inhibitors can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable prodrug” or “prodrug” represents those prodrugs of the modified serine proteinase inhibitors of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent protein having a structure of a disclosed modified serine proteinase inhibitor, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a protein disclosed herein) and whose structure is sufficiently similar to those disclosed herein and based upon that similarity, would be expected by one skilled in the art to exhibit the same or similar activities and utilities as the claimed modified serine proteinase inhibitors, or to induce, as a precursor, the same or similar activities and utilities as the claimed proteins. Exemplary derivatives include salts, esters, amides, salts of esters or amides, and N-oxides of a parent protein.

The term “contacting” as used herein refers to bringing a disclosed modified serine proteinase inhibitor or pharmaceutical composition in proximity to a cell, a target protein, or other biological entity together in such a manner that the disclosed protein or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

The disclosed modified serine proteinase inhibitors can be used in the form of salts derived from inorganic or organic acids. Pharmaceutically acceptable salts include salts of acidic or basic groups present in the disclosed modified serine proteinase inhibitors. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the parent protein with a suitable pharmaceutically acceptable base. The salts can be prepared in situ during the final isolation and purification of the proteins of the present disclosure; or following final isolation by reacting a free base function, such as a secondary or tertiary amine, of a disclosed protein with a suitable inorganic or organic acid; or reacting a free acid function, such as a carboxylic acid, of a disclosed modified serine proteinase inhibitor with a suitable inorganic or organic base.

Acidic addition salts can be prepared in situ during the final isolation and purification of a disclosed protein, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed protein, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

In various aspects, the present disclosure relates to pharmaceutical compositions comprising a therapeutically effective amount of at least one disclosed modified serine proteinase inhibitor or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed modified serine proteinase inhibitor or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, intravitreal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed protein, a pharmaceutically acceptable salt, a hydrate thereof, a solvate thereof, a polymorph thereof, or a stereochemically isomeric form thereof. In a further aspect, a disclosed protein a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed protein are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms that the disclosed proteins are able to form.

In various aspects, a disclosed protein comprising an acidic group or moiety, e.g., a carboxylic acid group, can be used to prepare a pharmaceutically acceptable salt. For example, such a disclosed protein may comprise an isolation step comprising treatment with a suitable inorganic or organic base. In some cases, it may be desirable in practice to initially isolate a protein from the cell culture as a pharmaceutically unacceptable salt and then simply convert the latter back to the free acid form by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed modified serine proteinase inhibitor comprising a protonatable group or moiety, e.g., an amino group, can be used to prepare a pharmaceutically acceptable salt. For example, such a disclosed protein may comprise an isolation step comprising treatment with a suitable inorganic or organic acid. In some cases, it may be desirable in practice to initially isolate a protein from the reaction mixture as a pharmaceutically unacceptable salt and then simply convert the latter back to the free base form by treatment with a basic reagent, and subsequently convert the free base to a pharmaceutically acceptable acid addition salt. These acid addition salts can be readily prepared using conventional techniques, e.g., by treating the corresponding basic proteins with an aqueous solution containing the desired pharmacologically acceptable anions and then evaporating the resulting solution to dryness, preferably under reduced pressure. Alternatively, they also can be prepared by treating the free base form of the disclosed modified serine proteinase inhibitor with a suitable pharmaceutically acceptable non-toxic inorganic or organic acid.

Acids which can be used to prepare the pharmaceutically acceptable acid-addition salts of the base forms are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the modified serine proteinase inhibitors of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the proteins of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a modified serine proteinase inhibitor of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed protein, or a pharmaceutically acceptable salt thereof. In a further aspect, a disclosed protein, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The modified serine proteinase inhibitors described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The proteins may be administered as a dosage that has a known quantity of the proteins.

Various dosage forms may be suitable depending upon clinical population (e.g., age and severity of clinical condition), solubility properties of the specific disclosed modified serine proteinase inhibitor used, and the like. Accordingly, the disclosed proteins can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C0-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed protein that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed modified serine proteinase inhibitor can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik and angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed protein in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the modified serine proteinase inhibitors according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed proteins can also be formulated as a depot preparation. Such long-acting formulations can be administered by implantation (e.g., subcutaneously or intramuscularly) or by intramuscular injection. Thus, for example, the proteins can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a modified serine proteinase inhibitor of the present disclosure, or pharmaceutically acceptable salts thereof, via conventional processing methods. As an example, a cream or ointment is prepared by mixing hydrophilic material and water, together with about 5 wt % to about 10 wt % of the protein, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a protein of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed protein formulated in a compatible pharmaceutical carrier may also be prepared, placed in an appropriate container, and labeled for treatment of an indicated condition.

The exact dosage and frequency of administration depends on the particular disclosed modified serine proteinase inhibitor, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the modified serine proteinase inhibitors of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment of conditions that require modulation of serine proteinase inhibitor activity, an appropriate dosage level can, in one aspect, be fixed. In one aspect, the dosage can be from about 0.1 to about 1000 mg, or can be about 0.1, 1, 5, 10, 25, 50, 75, 100, 150, 200, 250, 300, 350, 400, 450, 500, 550, 600, 650, 700, 750, 800, 850, 900, 950, or about 1000 mg, or a combination of any of the foregoing values, or a range encompassing any of the foregoing values. In one aspect, the dosage can be about 200 mg. In another aspect, the fixed dosage can be administered by a method such as, for example, intravenous infusion, at fixed time intervals. In one aspect, the fixed time interval can be about once per week, about once every other week, or about once every three weeks. In some aspects, various concentrations and amounts of the fixed dosage form can be supplied. In one aspect, the concentration can be 80 mg/0.8 mL, or 40 mg/0.4 mL, or 0.1 mg/0.2 mL, or 0.125 mg/0.1 mL, or 0.3 mg/0.05 mL, or 0.6 mg/0.05 mL, or 2 mg/0.05 mL.

In an alternative aspect, an appropriate dosage level can generally be from about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. In some aspects, dosage can vary based on patient sex (e.g., men may receive 2.5 mg/0.5 mL while women may receive 5 mg/l mL). For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The modified serine proteinase inhibitor can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific protein employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

### Methods for Treating Diseases and Health Conditions

In one aspect, disclosed herein is a method for treating a condition relating to increased vascular permeability, a condition relating to increased angiogenesis, an ocular disease, a condition associated with reduced bone mass, skin aging, a cancer, a wound, or a condition associated with inflammation in a subject, the method including the steps of administering to the subject the modified serine proteinase inhibitor disclosed herein or a pharmaceutical composition including the modified serine proteinase inhibitor.

In one aspect, the condition associated with increased vascular permeability or increased angiogenesis includes, but is not limited to, sepsis acute respiratory distress syndrome, nephrotic syndrome, diabetic neuropathy, preproliferative diabetic retinopathy, hemangioma, Osler-Webber syndrome, plaque neovascularization, telangiectasia, angiofibroma, wound granularization, nasal polyps, and benign neoplasias.

In another aspect, the cancer can be leukemia, prostate cancer, breast cancer, ovarian cancer, melanoma or metatstatic melanoma, bone cancer, or the like.

In still another aspect, the ocular disease can be dry eye, keratopathy induced by contact lens or radiation, Stevens Johnson syndrome, aniridia, a limbal tumor, ocular cicatricial pemphigoid, corneal neovascularization, diabetic retinopathy, wet type acute macular degeneration, or a corneal wound caused by pterygium, recurrent corneal erosion, chemical or thermal burn, herpes virus, or the like.

In one aspect, the condition associated with reduced bone mass can include osteogenesis imperfecta, osteoporosis, osteoarthritis, bone fracture, or the like.

In yet another aspect, the modified serine proteinase inhibitors useful herein may be used to promote wound healing such as, for example, healing of epithelial wounds caused by surgical excision, infection, chemical or thermal burns, skin graft donor sites, bedsores, ischemic necrosis, or diabetes-related wounds.

In a further aspect, the modified serine proteinase inhibitors useful herein can be used to treat a medical condition of the female reproductive system such as, for example, ovarian hyperstimulation syndrome, endometriosis, infertility, polycystic ovarian syndrome, or the like.

In one aspect, the modified serine proteinase inhibitors disclosed herein can be used to promote collagen synthesis and/or to promote dermal fibroblast proliferation. Further in this aspect, the modified serine proteinase inhibitors disclosed herein can be used to treat skin aging including, but not limited to, photoaging or UV-induced aging.

In a further aspect, the modified serine proteinase inhibitors disclosed herein can be used to treat any condition related to serine proteinase activity including, but not limited to, elastosis, vascular disorders involving fibrinoid formation, coagulation disorders, arteriosclerosis, ischemia, arthroses, diabetes, emphysema, septic shock, lung diseases, excessive complement activation, ulcers, ulcerative colitis, pancreatitis, psoriasis, fibrinolytic disease, arthropathy, hypertension, congestive heart failure, cirrhosis, allergy caused by proteases, and the like. In another aspect, the modified serine proteinase inhibitors are useful as anti-coagulants, anti-thrombotics, anti-microbial agents, anti-fungal agents, anti-parasitic agents, and the like.

In yet another aspect, the modified serine proteinase inhibitors disclosed herein can be used to treat conditions related to inflammation, cell death, vascular leakage, tumor growth, apoptosis, cytoskeletal function, mitochondrial function, oxidative stress, angiogenesis, neurogenesis, cell growth, immune function, cell differentiation, adipogenesis, bone deposition, or gene expression.

In one aspect, the modified serine proteinase inhibitors disclosed herein can be expressed in host cells, purified, and administered to subjects systemically or locally as components of pharmaceutical compositions. In an alternative aspect, therapies for the indicated conditions can be gene-based. Further in this aspect, engineered serine proteinase inhibitor mRNA can be delivered by a synthetic vehicle or a physical method such as, for example, electroporation. In another aspect, engineered serine proteinase inhibitor cDNA can be transported by a viral vector or a plasmid.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

Aspect 1: A modified serine proteinase inhibitor comprising anionic amino acid residues and cationic amino acid residues, wherein the sum of the cationic residues is greater than the sum of the anionic residues in the modified serine proteinase inhibitor.

Aspect 2: The modified serine proteinase inhibitor of aspect 1, wherein one or more amino acid residues of the corresponding naturally-occurring serine proteinase inhibitor are replaced with a cationic amino acid residue.

Aspect 3: The modified serine proteinase inhibitor of aspect 1, wherein one or more anionic amino acid residues of the corresponding naturally-occurring serine proteinase inhibitor are replaced with a cationic amino acid residue.

Aspect 4: The modified serine proteinase inhibitor of aspect 3, wherein the one or more anionic amino acid residues replaced with the cationic amino acid residue are present between s2A and hE of the naturally-occurring serine proteinase inhibitor, between hF and s3A of the naturally-occurring serine proteinase inhibitor, or a combination thereof.

Aspect 5: The modified serine proteinase inhibitor of aspect 1, wherein one or more anionic amino acid residues of the corresponding naturally-occurring serine proteinase inhibitor are removed.

Aspect 6: The modified serine proteinase inhibitor of aspect 5, wherein the one or more anionic amino acid residues are present between s2A and hE of the naturally-occurring serine proteinase inhibitor, between hF and s3A of the naturally-occurring serine proteinase inhibitor, or a combination thereof are removed.

Aspect 7: The modified serine proteinase inhibitor of aspect 1, wherein one or more cationic acid residues are added to the corresponding naturally-occurring serine proteinase inhibitor.

Aspect 8: The modified serine proteinase inhibitor of aspect 7, wherein the one or more cationic amino acid residues are added between s2A and hE of the naturally-occurring serine proteinase inhibitor, between hF and s3A of the naturally-occurring serine proteinase inhibitor, or a combination thereof.

Aspect 9: The modified serine proteinase inhibitor in any one of aspects 1 to 8, wherein the naturally-occurring serine proteinase inhibitor comprises alpha 1-antitrypsin, C1 esterase Inhibitor, antithrombin, plasminogen activator inhibitor, pigment epithelium-derived factor, or a combination thereof.

Aspect 10: The modified serine proteinase inhibitor in any one of aspects 1 to 9, wherein the cationic amino acid residue comprises lysine, arginine, histidine, or any combination thereof.

Aspect 11: The modified serine proteinase inhibitor in any one of claims 1 to 10, wherein the anionic amino acid residue comprises aspartic acid, glutamic acid, or a combination thereof.

Aspect 12: The modified serine proteinase inhibitor in any one of aspects 1 to 11, wherein the modified serine proteinase inhibitor has an isoelectric point (pI) that is from 10% to 90% greater than the naturally-occurring serine proteinase inhibitor.

Aspect 13: The modified serine proteinase inhibitor in any one of aspects 1 to 12, wherein the sum of the lysine and arginine residues is greater than the sum of the aspartate and glutamate residues in the modified serine proteinase inhibitor.

Aspect 14: The modified serine proteinase inhibitor in any one of aspects 1 to 13, wherein the sum of the lysine and arginine residues is from 46 to 55.

Aspect 15: The modified serine proteinase inhibitor in any one of aspects 1 to 14, wherein the modified serine proteinase inhibitor is pigment epithelium derived factor (PEDF), and wherein the sum of the cationic amino acid residues present between hF and s3A is greater than the sum of anionic amino acid residues present between hF and s3A.

Aspect 16: The modified serine proteinase inhibitor in any one of aspects 1 to 15, wherein the modified serine proteinase inhibitor is pigment epithelium derived factor (PEDF), wherein the sum of the cationic amino acid residues present between s2A and hE is greater than the sum of anionic amino acid residues present between s2A and hE.

Aspect 17: The modified serine proteinase inhibitor of SEQ ID NO. 5, 7, 9, 11, 13, or a combination thereof.

Aspect 18: A method for treating a condition relating to increased vascular permeability, a condition relating to increased angiogenesis, an ocular disease, a condition associated with reduced bone mass, skin aging, a wound, a cancer, or a condition associated with inflammation in a subject, the method comprising administering to the subject the modified serine proteinase inhibitor in any one of aspects 1 to 17.

Aspect 19: A DNA construct comprising SEQ ID NO. 4, 6, 8, 10, or 12 or at least 70% homology thereto.

Aspect 20: A vector comprising the DNA construct of aspect 19.

Aspect 21: The vector of aspect 20, wherein the vector is a plasmid.

Aspect 22: The vector of aspect 21, wherein the plasmid comprises pWLneo, pSV2cat, pOG44, pXT1, pSG, pSVK3, pBSK, pBSKII, pYES, pYES2, pUC, pUC19, pETDuet-1, p3xFLAG, pBApo, pBI, pcDNA, pCEP, pCI, pCMV, pCTAP, pDEST, pEF, pFLAG, pFN, phCMV, pHet, pIRES, pmRi, pNTAP, prHom, pSecTag, pT-Rex, pTet, pTracer, pTRE, pVAX, pX330, pZeoSV2, a derivative or variant thereof, or a combination thereof.

Aspect 23: The vector of aspect 20, wherein the vector comprises an adenovirus or an adeno-associated virus.

Aspect 24: A vector comprising RNA, wherein the RNA is complementary to the DNA construct of aspect 19.

Aspect 25: The vector of aspect 24, wherein the vector comprises a lentivirus or Sendai virus.

Aspect 26: The vector of aspect 24, wherein the vector comprises mRNA.

Aspect 27: A biological device comprising host cells transformed with the DNA construct of aspect 19 or the vector of any of aspects 20-26.

Aspect 28: A modified serine proteinase inhibitor produced by culturing the biological device of aspect 27.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Engineering the PEDF Sequence

A wild type cDNA having SEQ ID NO. 1 or SEQ ID NO. 2 and encoding for a protein with SEQ ID NO. 3 was used as a control. Selected nucleobases from SEQ ID NO. 2 were then altered to generate codons for increasing numbers of lysine residues, which thereby resulted in increasing levels of positive charge density on the engineered PEDF surface. Underlined residues in Table 1 below were mutated:

Table 1
Sequences
SEQ ID NO. 1
ATGCAGGCCCTGGTGCTACTCCTCTGCATTGGAGCCCTCCTCGGGCACAGCAGCTGCCAG
AACCCTGCCAGCCCCCCGGAGGAGGGCTCCCCAGACCCCGACAGCACAGGGGCGCTGGTG
GAGGAGGAGGATCCTTTCTTCAAAGTCCCCGTGAACAAGCTGGCAGCGGCTGTCTCCAAC
TTCGGCTATGACCTGTACCGGGTGCGATCCAGCACGAGCCCCACGACCAACGTGCTCCTG
TCTCCTCTCAGTGTGGCCACGGCCCTCTCGGCCCTCTCGCTGGGAGCGGANNAGCGAACA
GAATCCATCATTCACCGGGCTCTCTACTATGACTTGATCAGCAGCCCAGACATCCATGGT
ACCTATAAGGAGCTCCTTGACACGGTCACTGCCCCCCAGAAGAACCTCAAGAGTGCCTCC
CGGATCGTCTTTGAGAAGAAGCTGCGCATAAAATCCAGCTTTGTGGCACCTCTGGAAAAG
TCATATGGGACCAGGCCCAGAGTCCTGACGGGCAACCCTCGCTTGGACCTGCAAGAGATC
AACAACTGGGTGCAGGCGCAGATGAAAGGGAAGCTCGCCAGGTCCACAAAGGAAATTCCC
GATGAGATCAGCATTCTCCTTCTCGGTGTGGCGCACTTCAAGGGGCAGTGGGTAACAAAG
TTTGACTCCAGAAAGACTTCCCTCGAGGATTTCTACTTGGATGAAGAGAGGACCGTGAGG
GTCCCCATGATGTCGGACCCTAAGGCTGTTTTACGCTATGGCTTGGATTCAGATCTCAGC
TGCAAGATTGCCCAGCTGCCCTTGACCGGAAGCATGAGTATCATCTTCTTCCTGCCCCTG
AAAGTGACCCAGAATTTGACCTTGATAGAGGAGAGCCTCACCTCCGAGTTCATTCATGAC
ATAGACCGAGAACTGAAGACCGTGCAGGCGGTCCTCACTGTCCCCAAGCTGAAGCTGAGT
TATGAAGGCGAAGTCACCAAGTCCCTGCAGGAGATGAAGCTGCAATCCTTGTTTGATTCA
CCAGACTTTAGCAAGATCACAGGCAAACCCATCAAGCTGACTCAGGTGGAACACCGGGCT
GGCTTTGAGTGGAACGAGGATGGGGCGGGAACCACCCCCAGCCCAGGGCTGCAGCCTGCC
CACCTCACCTTCCCGCTGGACTATCACCTTAACCAGCCTTTCATCTTCGTACTGAGGGAC
ACAGACACAGGGGCCCTTCTCTTCATTGGCAAGATTCTGGACCCCAGGGGCCCCTAA
SEQ ID NO. 2
ATGCAGGCCCTGGTGCTGCTGCTGTGCATTGGTGCCCTGCTGGGTCATAGTAGTTGTCAG
AATCCCGCTAGTCCTCCCGAAGAAGGCTCTCCCGACCCTGATAGCACCGGCGCCCTGGTG
GAGGAGGAGGACCCTTTCTTTAAGGTGCCAGTGAACAAGCTGGCCGCTGCCGTGTCTAAT
TTCGGCTACGATCTGTATCGGGTGAGGAGCTCCACAAGCCCCACCACAAACGTGCTGCTG
TCCCCTCTGTCTGTGGCTACCGCCCTGAGCGCCCTGAGCCTGGGAGCTGAGCAGAGAACA
GAGTCCATCATCCACCGCGCTCTGTACTATGACCTGATCTCTAGCCCCGATATCCACGGC
ACCTACAAGGAGCTGCTGGACACCGTGACAGCTCCTCAGAAGAACCTGAAGAGCGCCTCC
CGGATCGTGTTCGAGAAGAAGCTGAGGATCAAGTCCTCTTTTGTGGCCCCACTGGAGAAG
TCTTATGGCACCAGACCTCGCGTGCTGACAGGCAATCCAAGACTGGATCTGCAGGAGATC
AACAATTGGGTGCAGGCTCAGATGAAGGGCAAGCTGGCCCGCAGCACCAAGGAGATCCCC
GACGAGATCTCCATCCTGCTGCTGGGCGTGGCTCACTTCAAGGGCCAGTGGGTGACCAAG
TTTGATAGCCGGAAGACATCCCTGGAGGACTTCTACCTGGATGAGGAGCGGACAGTGAGG
GTGCCCATGATGTCCGACCCTAAGGCTGTGCTGAGATATGGCCTGGACTCTGATCTGAGC
TGCAAGATCGCCCAGCTGCCTCTGACCGGCTCTATGAGCATCATCTTCTTTCTGCCACTG
AAGGTGACCCAGAATCTGACACTGATCGAGGAGTCCCTGACATCTGAGTTTATCCACGAC
ATCGATCGGGAGCTGAAGACCGTGCAGGCCGTGCTGACAGTGCCTAAGCTGAAGCTGTCC
TACGAGGGCGAGGTGACCAAGTCTCTGCAGGAGATGAAGCTGCAGTCTCTGTTCGACAGC
CCAGATTTTTCCAAGATCACCGGCAAGCCCATCAAGCTGACACAGGTGGAGCACAGAGCT
GGATTCGAGTGGAACGAGGACGGAGCTGGAACCACACCAAGCCCAGGCCTGCAGCCAGCT
CACCTGACCTTTCCCCTGGATTATCACCTGAATCAGCCCTTCATCTTTGTGCTGCGCGAC
ACCGATACAGGCGCCCTGCTGTTTATCGGCAAGATCCTGGACCCTCGGGGACCATAA
SEQ ID NO. 3
MQALVLLLCIGALLGHSSCQNPASPPEEGSPDPDSTGALVEEEDPFFKVP
VNKLAAAVSNFGYDLYRVRSSTSPTTNVLLSPLSVATALSALSLGAEQRT
ESIIHRALYYDLISSPDIHGTYKELLDTVTAPQKNLKSASRIVFEKKLRI
KSSFVAPLEKSYGTRPRVLTGNPRLDLQEINNWVQAQMKGKLARSTKEIP
DEISILLLGVAHFKGQWVTKFDSRKTSLEDFYLDEERTVRVPMMSDPKAV
LRYGLDSDLSCKIAQLPLTGSMSIIFFLPLKVTQNLTLIEESLTSEFIHD
IDRELKTVQAVLTVPKLKLSYEGEVTKSLQEMKLQSLFDSPDFSKITGKP
IKLTQVEHRAGFEWNEDGAGTTPSPGLQPAHLTFPLDYHLNQPFIFVLRD
TDTGALLFIGKILDPRGP

| cDNA SEQ ID NO. | cDNA Sequence | Protein SEQ ID NO. | Protein Sequence |
|---|---|---|---|
| 4 | ATGCAGGCCCTGGTGCTGCTGCTGTGCATTGGTGCCCTGCTGGGTCATAGTAGTTGTCAGAATCC | 5 | MQALVLLLCIGALLGHSSCQNPASPPEEGSPD |
| | CGCTAGTCCTCCCGAAGAAGGCTCTCCCGACCCTGATAGCACCGGCGCCCTGGTGGAGGAGGAGG | | PDSTGALVEEEDPFFKVPVNKLAAAVSNFGYD |
| | ACCCTTTCTTTAAGGTGCCAGTGAACAAGCTGGCCGCTGCCGTGTCTAATTTCGGCTACGATCTG | | LYRVRSSTSPTTNVLLSPLSVATALSALSLGA |
| | TATCGGGTGAGGAGCTCCACAAGCCCCACCACAAACGTGCTGCTGTCCCCTCTGTCTGTGGCTAC | | EQRTESIIHRALYYDLISSPDIHGTYKELLDT |
| | CGCCCTGAGCGCCCTGAGCCTGGGAGCTGAGCAGAGAACAGAGTCCATCATCCACCGCGCTCTGT | | VTAPQKNLKSASRIVFEKKLRIKSSFVAPLEK |
| | ACTATGACCTGATCTCTAGCCCCGATATCCACGGCACCTACAAGGAGCTGCTGGACACCGTGACA | | SYGTRPRVLTGNPRLDLQEINNWVQAQMKGKL |
| | GCTCCTCAGAAGAACCTGAAGAGCGCCTCCCGGATCGTGTTCGAGAAGAAGCTGAGGATCAAGTC | | ARSTKKIPKKISILLLGVAHFKGQWVTKFDSR |
| | CTCTTTTGTGGCCCCACTGGAGAAGTCTTATGGCACCAGACCTCGCGTGCTGACAGGCAATCCAA | | KTSLEDFYLDEERTVRVPMMSDPKAVLRYGLD |
| | GACTGGATCTGCAGGAGATCAACAATTGGGTGCAGGCTCAGATGAAGGGCAAGCTGGCCCGCAGC | | SDLSCKIAQLPLTGSMSIIFFLPLKVTQNLTL |
| | ACCAAGAAGATCCCCAAGAAGATCTCCATCCTGCTGCTGGGCGTGGCTCACTTCAAGGGCCAGTG | | IEESLTSEFIHDIDRELKTVQAVLTVPKLKLS |
| | GGTGACCAAGTTTGATAGCCGGAAGACATCCCTGGAGGACTTCTACCTGGATGAGGAGCGGACAG | | YEGEVTKSLQEMKLQSLFDSPDFSKITGKPIK |
| | TGAGGGTGCCCATGATGTCCGACCCTAAGGCTGTGCTGAGATATGGCCTGGACTCTGATCTGAGC | | LTQVEHRAGFEWNEDGAGTTPSPGLQPAHLTF |
| | TGCAAGATCGCCCAGCTGCCTCTGACCGGCTCTATGAGCATCATCTTCTTTCTGCCACTGAAGGT | | PLDYHLNQPFIFVLRDTDTGALLFIGKILDPR |
| | GACCCAGAATCTGACACTGATCGAGGAGTCCCTGACATCTGAGTTTATCCACGACATCGATCGGG | | GP |
| | AGCTGAAGACCGTGCAGGCCGTGCTGACAGTGCCTAAGCTGAAGCTGTCCTACGAGGGCGAGGTG | | |
| | ACCAAGTCTCTGCAGGAGATGAAGCTGCAGTCTCTGTTCGACAGCCCAGATTTTTCCAAGATCAC | | |
| | CGGCAAGCCCATCAAGCTGACACAGGTGGAGCACAGAGCTGGATTCGAGTGGAACGAGGACGGAG | | |
| | CTGGAACCACACCAAGCCCAGGCCTGCAGCCAGCTCACCTGACCTTTCCCCTGGATTATCACCTG | | |
| | AATCAGCCCTTCATCTTTGTGCTGCGCGACACCGATACAGGCGCCCTGCTGTTTATCGGCAAGAT | | |
| | CCTGGACCCTCGGGGACCATAA | | |
| 6 | ATGCAGGCCCTGGTGCTGCTGCTGTGCATTGGTGCCCTGCTGGGTCATAGTAGTTGTCAGAATCC | 7 | MQALVLLLCIGALLGHSSCQNPASPPEEGSPD |
| | CGCTAGTCCTCCCGAAGAAGGCTCTCCCGACCCTGATAGCACCGGCGCCCTGGTGGAGGAGGAGG | | PDSTGALVEEEDPFFKVPVNKLAAAVSNFGYD |
| | ACCCTTTCTTTAAGGTGCCAGTGAACAAGCTGGCCGCTGCCGTGTCTAATTTCGGCTACGATCTG | | LYRVRSSTSPTTNVLLSPLSVATALSALSLGA |
| | TATCGGGTGAGGAGCTCCACAAGCCCCACCACAAACGTGCTGCTGTCCCCTCTGTCTGTGGCTAC | | EQRTESIIHRALYYDLISSPDIHGTYKELLDT |
| | CGCCCTGAGCGCCCTGAGCCTGGGAGCTGAGCAGAGAACAGAGTCCATCATCCACCGCGCTCTGT | | VTAPQKNLKSASRIVFEKKLRIKSSFVAPLEK |
| | ACTATGACCTGATCTCTAGCCCCGATATCCACGGCACCTACAAGGAGCTGCTGGACACCGTGACA | | SYGTRPRVLTGNPRLDLQEINNWVQAQMKGKL |
| | GCTCCTCAGAAGAACCTGAAGAGCGCCTCCCGGATCGTGTTCGAGAAGAAGCTGAGGATCAAGTC | | KRSKKKIPKKISILLLGVAHFKGQWVTKFDSR |
| | CTCTTTTGTGGCCCCACTGGAGAAGTCTTATGGCACCAGACCTCGCGTGCTGACAGGCAATCCAA | | KTSLEDFYLDEERTVRVPMMSDPKAVLRYGLD |
| | GACTGGATCTGCAGGAGATCAACAATTGGGTGCAGGCTCAGATGAAGGGCAAGCTGAAGCGCAGC | | SDLSCKIAQLPLTGSMSIIFFLPLKVTQNLTL |
| | AAGAAGAAGATCCCCAAGAAGATCTCCATCCTGCTGCTGGGCGTGGCTCACTTCAAGGGCCAGTG | | IEESLTSEFIHDIDRELKTVQAVLTVPKLKLS |
| | GGTGACCAAGTTTGATAGCCGGAAGACATCCCTGGAGGACTTCTACCTGGATGAGGAGCGGACAG | | YEGEVTKSLQEMKLQSLFDSPDFSKITGKPIK |
| | TGAGGGTGCCCATGATGTCCGACCCTAAGGCTGTGCTGAGATATGGCCTGGACTCTGATCTGAGC | | LTQVEHRAGFEWNEDGAGTTPSPGLQPAHLTF |
| | TGCAAGATCGCCCAGCTGCCTCTGACCGGCTCTATGAGCATCATCTTCTTTCTGCCACTGAAGGT | | PLDYHLNQPFIFVLRDTDTGALLFIGKILDPR |
| | GACCCAGAATCTGACACTGATCGAGGAGTCCCTGACATCTGAGTTTATCCACGACATCGATCGGG | | GP |
| | AGCTGAAGACCGTGCAGGCCGTGCTGACAGTGCCTAAGCTGAAGCTGTCCTACGAGGGCGAGGTG | | |
| | ACCAAGTCTCTGCAGGAGATGAAGCTGCAGTCTCTGTTCGACAGCCCAGATTTTTCCAAGATCAC | | |
| | CGGCAAGCCCATCAAGCTGACACAGGTGGAGCACAGAGCTGGATTCGAGTGGAACGAGGACGGAG | | |
| | CTGGAACCACACCAAGCCCAGGCCTGCAGCCAGCTCACCTGACCTTTCCCCTGGATTATCACCTG | | |
| | AATCAGCCCTTCATCTTTGTGCTGCGCGACACCGATACAGGCGCCCTGCTGTTTATCGGCAAGAT | | |
| | CCTGGACCCTCGGGGACCATAA | | |
| 8 | ATGCAGGCCCTGGTGCTGCTGCTGTGCATTGGTGCCCTGCTGGGTCATAGTAGTTGTCAGAATCC | 9 | MQALVLLLCIGALLGHSSCQNPASPPEEGSPD |
| | CGCTAGTCCTCCCGAAGAAGGCTCTCCCGACCCTGATAGCACCGGCGCCCTGGTGGAGGAGGAGG | | PDSTGALVEEEDPFFKVPVNKLAAAVSNFGYD |
| | ACCCTTTCTTTAAGGTGCCAGTGAACAAGCTGGCCGCTGCCGTGTCTAATTTCGGCTACGATCTG | | LYRVRSSTSPTTNVLLSPLSVATALSALSLGA |
| | TATCGGGTGAGGAGCTCCACAAGCCCCACCACAAACGTGCTGCTGTCCCCTCTGTCTGTGGCTAC | | EQRTESIIHRALYYDLISSPDIHGTYKELLDT |
| | CGCCCTGAGCGCCCTGAGCCTGGGAGCTGAGCAGAGAACAGAGTCCATCATCCACCGCGCTCTGT | | VTAPQKNLKSASRIVFEKKLRIKSSFVAPLEK |
| | ACTATGACCTGATCTCTAGCCCCGATATCCACGGCACCTACAAGGAGCTGCTGGACACCGTGACA | | SYGTRPRVLTKNPRLDLQEINNWVQKQMKGKL |
| | GCTCCTCAGAAGAACCTGAAGAGCGCCTCCCGGATCGTGTTCGAGAAGAAGCTGAGGATCAAGTC | | KRSTKEIPKRISILLLGVAHFKGQWVTKFDSR |
| | CTCTTTTGTGGCCCCACTGGAGAAGTCTTATGGCACCAGACCTCGCGTGCTGACAAAGAATCCAA | | KTSLEDFYLDEERTVRVPMMSDPKAVLRYGLD |
| | GACTGGATCTGCAGGAGATCAACAATTGGGTGCAGAAGCAGATGAAGGGCAAGCTGAAGCGCAGC | | SDLSCKIAQLPLTGSMSIIFFLPLKVTQNLTL |
| | ACCAAGGAGATCCCCAAGCGGATCTCCATCCTGCTGCTGGGCGTGGCTCACTTCAAGGGCCAGTG | | IEESLTSEFIHDIDRELKTVQAVLTVPKLKLS |
| | GGTGACCAAGTTTGATAGCCGGAAGACATCCCTGGAGGACTTCTACCTGGATGAGGAGCGGACAG | | YEGEVTKSLQEMKLQSLFDSPDFSKITGKPIK |
| | TGAGGGTGCCCATGATGTCCGACCCTAAGGCTGTGCTGAGATATGGCCTGGACTCTGATCTGAGC | | LTQVEHRAGFEWNEDGAGTTPSPGLQPAHLTF |
| | TGCAAGATCGCCCAGCTGCCTCTGACCGGCTCTATGAGCATCATCTTCTTTCTGCCACTGAAGGT | | PLDYHLNQPFIFVLRDTDTGALLFIGKILDPR |
| | GACCCAGAATCTGACACTGATCGAGGAGTCCCTGACATCTGAGTTTATCCACGACATCGATCGGG | | GP |
| | AGCTGAAGACCGTGCAGGCCGTGCTGACAGTGCCTAAGCTGAAGCTGTCCTACGAGGGCGAGGTG | | |
| | ACCAAGTCTCTGCAGGAGATGAAGCTGCAGTCTCTGTTCGACAGCCCAGATTTTTCCAAGATCAC | | |
| | CGGCAAGCCCATCAAGCTGACACAGGTGGAGCACAGAGCTGGATTCGAGTGGAACGAGGACGGAG | | |
| | CTGGAACCACACCAAGCCCAGGCCTGCAGCCAGCTCACCTGACCTTTCCCCTGGATTATCACCTG | | |
| | AATCAGCCCTTCATCTTTGTGCTGCGCGACACCGATACAGGCGCCCTGCTGTTTATCGGCAAGAT | | |
| | CCTGGACCCTCGGGGACCATAA | | |
| 10 | ATGCAGGCCCTGGTGCTGCTGCTGTGCATTGGTGCCCTGCTGGGTCATAGTAGTTGTCAGAATCC | 11 | MQALVLLLCIGALLGHSSCQNPASPPEEGSPD |
| | CGCTAGTCCTCCCGAAGAAGGCTCTCCCGACCCTGATAGCACCGGCGCCCTGGTGGAGGAGGAGG | | PDSTGALVEEEDPFFKVPVNKLAAAVSNFGYD |
| | ACCCTTTCTTTAAGGTGCCAGTGAACAAGCTGGCCGCTGCCGTGTCTAATTTCGGCTACGATCTG | | LYRVRSSTSPTTNVLLSPLSVATALSALSLGA |
| | TATCGGGTGAGGAGCTCCACAAGCCCCACCACAAACGTGCTGCTGTCCCCTCTGTCTGTGGCTAC | | EQRTESIIHRALYYDLISSPDIHGTYKELLDT |
| | CGCCCTGAGCGCCCTGAGCCTGGGAGCTGAGCAGAGAACAGAGTCCATCATCCACCGCGCTCTGT | | VTAPQKNLKSASRIVFEKKLRIKSSFVAPLEK |
| | ACTATGACCTGATCTCTAGCCCCGATATCCACGGCACCTACAAGGAGCTGCTGGACACCGTGACA | | SYGTRPRVLTKNPRLDLQEINNWVQKQMKGKL |
| | GCTCCTCAGAAGAACCTGAAGAGCGCCTCCCGGATCGTGTTCGAGAAGAAGCTGAGGATCAAGTC | | KRSTKKIPKKISILLLGVAHFKGQWVTKFDSR |
| | CTCTTTTGTGGCCCCACTGGAGAAGTCTTATGGCACCAGACCTCGCGTGCTGACAAAGAATCCAA | | KTSLEDFYLDEERTVRVPMMSDPKAVLRYGLD |
| | GACTGGATCTGCAGGAGATCAACAATTGGGTGCAGAAGCAGATGAAGGGCAAGCTGAAGCGCAGC | | SDLSCKIAQLPLTGSMSIIFFLPLKVTQNLTL |
| | ACCAAGAAGATCCCCAAGAAGATCTCCATCCTGCTGCTGGGCGTGGCTCACTTCAAGGGCCAGTG | | IEESLTSEFIHDIDRELKTVQAVLTVPKLKLS |
| | GGTGACCAAGTTTGATAGCCGGAAGACATCCCTGGAGGACTTCTACCTGGATGAGGAGCGGACAG | | YEGEVTKSLQEMKLQSLFDSPDFSKITGKPIK |
| | TGAGGGTGCCCATGATGTCCGACCCTAAGGCTGTGCTGAGATATGGCCTGGACTCTGATCTGAGC | | LTQVEHRAGFEWNEDGAGTTPSPGLQPAHLTF |
| | TGCAAGATCGCCCAGCTGCCTCTGACCGGCTCTATGAGCATCATCTTCTTTCTGCCACTGAAGGT | | PLDYHLNQPFIFVLRDTDTGALLFIGKILDPR |
| | GACCCAGAATCTGACACTGATCGAGGAGTCCCTGACATCTGAGTTTATCCACGACATCGATCGGG | | GP |
| | AGCTGAAGACCGTGCAGGCCGTGCTGACAGTGCCTAAGCTGAAGCTGTCCTACGAGGGCGAGGTG | | |
| | ACCAAGTCTCTGCAGGAGATGAAGCTGCAGTCTCTGTTCGACAGCCCAGATTTTTCCAAGATCAC | | |
| | CGGCAAGCCCATCAAGCTGACACAGGTGGAGCACAGAGCTGGATTCGAGTGGAACGAGGACGGAG | | |
| | CTGGAACCACACCAAGCCCAGGCCTGCAGCCAGCTCACCTGACCTTTCCCCTGGATTATCACCTG | | |
| | AATCAGCCCTTCATCTTTGTGCTGCGCGACACCGATACAGGCGCCCTGCTGTTTATCGGCAAGAT | | |
| | CCTGGACCCTCGGGGACCATAA | | |
| 12 | ATGCAGGCCCTGGTGCTGCTGCTGTGCATTGGTGCCCTGCTGGGTCATAGTAGTTGTCAGAATCC | 13 | MQALVLLLCIGALLGHSSCQNPASPPEEGSPD |
| | CGCTAGTCCTCCCGAAGAAGGCTCTCCCGACCCTGATAGCACCGGCGCCCTGGTGGAGGAGGAGG | | PDSTGALVEEEDPFFKVPVNKLAAAVSNFGYD |
| | ACCCTTTCTTTAAGGTGCCAGTGAACAAGCTGGCCGCTGCCGTGTCTAATTTCGGCTACGATCTG | | LYRVRSSTSPTTNVLLSPLSVATALSALSLGA |
| | TATCGGGTGAGGAGCTCCACAAGCCCCACCACAAACGTGCTGCTGTCCCCTCTGTCTGTGGCTAC | | EQRTESIIHRALYYDLISSPDIHGTYKELLDT |
| | CGCCCTGAGCGCCCTGAGCCTGGGAGCTGAGCAGAGAACAGAGTCCATCATCCACCGCGCTCTGT | | VTAPQKNLKSASRIVFEKKLRIKSSFVAPLKK |
| | ACTATGACCTGATCTCTAGCCCCGATATCCACGGCACCTACAAGGAGCTGCTGGACACCGTGACA | | SYGTRPRVLTKNPRLDLQEINNWVQKQMKGKL |
| | GCTCCTCAGAAGAACCTGAAGAGCGCCTCCCGGATCGTGTTCGAGAAGAAGCTGAGGATCAAGTC | | KRSTKKIPKKISILLLGVAHFKGQWVTKFDSR |
| | CTCTTTTGTGGCCCCACTGAAGAAGTCTTATGGCACCAGACCTCGCGTGCTGACAAAGAATCCAA | | KTSLEDFYLDEERTVRVPMMSDPKAVLRYGLD |
| | GACTGGATCTGCAGGAGATCAACAATTGGGTGCAGAAGCAGATGAAGGGCAAGCTGAAGCGCAGC | | SDLSCKIAQLPLTGSMSIIFFLPLKVTQNLTL |
| | ACCAAGAAGATCCCCAAGAAGATCTCCATCCTGCTGCTGGGCGTGGCTCACTTCAAGGGCCAGTG | | IEESLTSEFIHDIDRELKTVQAVLTVPKLKLS |
| | GGTGACCAAGTTTGATAGCCGGAAGACATCCCTGGAGGACTTCTACCTGGATGAGGAGCGGACAG | | YEGEVTKSLQEMKLQSLFDSPDFSKITGKPIK |
| | TGAGGGTGCCCATGATGTCCGACCCTAAGGCTGTGCTGAGATATGGCCTGGACTCTGATCTGAGC | | LTQVEHRAGFEWNEDGAGTTPSPGLQPAHLTF |
| | TGCAAGATCGCCCAGCTGCCTCTGACCGGCTCTATGAGCATCATCTTCTTTCTGCCACTGAAGGT | | PLDYHLNQPFIFVLRDTDTGALLFIGKILDPR |
| | GACCCAGAATCTGACACTGATCGAGGAGTCCCTGACATCTGAGTTTATCCACGACATCGATCGGG | | GP |
| | AGCTGAAGACCGTGCAGGCCGTGCTGACAGTGCCTAAGCTGAAGCTGTCCTACGAGGGCGAGGTG | | |
| | ACCAAGTCTCTGCAGGAGATGAAGCTGCAGTCTCTGTTCGACAGCCCAGATTTTTCCAAGATCAC | | |
| | CGGCAAGCCCATCAAGCTGACACAGGTGGAGCACAGAGCTGGATTCGAGTGGAACGAGGACGGAG | | |
| | CTGGAACCACACCAAGCCCAGGCCTGCAGCCAGCTCACCTGACCTTTCCCCTGGATTATCACCTG | | |
| | AATCAGCCCTTCATCTTTGTGCTGCGCGACACCGATACAGGCGCCCTGCTGTTTATCGGCAAGAT | | |
| | CCTGGACCCTCGGGGACCATAA | | |

### Example 2: Expression of Engineered PEDF

PEDF engineering began with the construction of plasmids encoding wild type human PEDF. Human PEDF cDNA was synthesized and codons were optimized to maximize protein production with the cDNA represented by SEQ ID NO. 2 providing the highest levels of expression. In some experiments, PEDF cDNA was obtained by isolation of a PEDF mRNA followed by reverse transcription and/or through a commercial vendor.

Following plasmid construction, human PEDF cDNA was cloned into an expression vector. The chosen plasmid allowed expression of PEDF in mammalian cells in order to ensure complete post-translational modification such as glycosylation. However, it was determined that the vector could be switched and PEDF expressed in E. coli, yeast, or insect cells.

Modifications of cDNA were achieved using site-directed mutagenesis in which PCR reactions created one or multiple mutations in the wild type cDNA sequence (that is, SEQ ID NO. 2). The mutations in the cDNAs were tailored to alter the amino acid sequence of PEDF. Alterations included the following: (1) addition, or incorporation of one or multiple amino acids not present in the wild type PEDF; (2) deletion, or removal of one or multiple amino acids present in the wild type PEDF; and (3) missense mutations, or conversions of one amino acid to a different amino acid. Following this, sequences of cDNAs encoding engineered PEDF were confirmed by Sanger sequencing and endotoxin-free plasmids were prepared.

Transient transfection was used to express engineered PEDF. Plasmids encoding engineered PEDF were introduced into mammalian cells using a chemical agent such as lipofectamine, polyethyleneimine, or the like. Here, human embryonic kidney cells (293T cell line) were transfected to maximize protein production, since the plasmid used included an SV40 origin of replication. Other common cell lines, including Chinese hamster ovary (CHO) and human embryonic kidney 293 (HEK293) cell lines were also used successfully in some experiments. 2-4 hours post transfection, the culture medium was replaced with serum-free medium and transfected cells were cultured for 2-3 days to produce PEDF. Both wild type and engineered PEDF are secretory proteins, and the culture medium was collected to harvest engineered PEDF.

Stable transfection was used to generate the cell lines that continuously produced engineered PEDF. Plasmids encoding engineered PEDF were first linearized by a restriction enzyme and introduced into mammalian cells using a transfection agent. 2-3 days later, the transfection medium was replaced with the fresh medium containing selective antibiotics such as puromycin and gentamicin, and cultured for 2-3 weeks until all cells became antibiotic-resistant. Production of PEDF was finally measured by western blotting or enzyme-linked immunosorbent assays.

### Example 3: Effect of PEDF Alterations

A comparison of structural and chemical features of wild type and engineered PEDF proteins used herein can be seen in Table 2:

TABLE 2
Properties of Wild Type and Modified PEDF Proteins

| PEDF | Wild type | 3 | 5.1 | 5.2 | 6 | 7 |
|---|---|---|---|---|---|---|
| cDNA SEQ ID NO. | 2 | 4 | 6 | 8 | 10 | 12 |
| Protein SEQ ID NO. | 3 | 5 | 7 | 9 | 11 | 13 |
| pI | 5.97 | 7.69 | 8.51 | 8.22 | 8.70 | 8.95 |
| Number of Lysine | 27 | 30 | 32 | 31 | 33 | 34 |
| Residues | | | | | | |
| Number of Arginine | 18 | 18 | 18 | 19 | 18 | 18 |
| Residues | | | | | | |
| Number of Aspartate | 24 | 23 | 23 | 23 | 23 | 23 |
| Residues | | | | | | |
| Number of Glutamate | 26 | 24 | 24 | 25 | 24 | 23 |
| Residues | | | | | | |

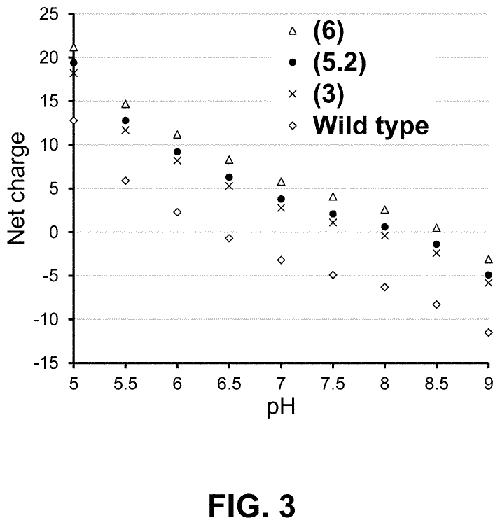

Isoelectric point (pI) was calculated using ExPASy (expert protein analysis system). Wild type PEDF carries more acidic residues (aspartate and glutamate) than basic residues (lysine and arginine). Following modification, engineered PEDF carries more basic residues than acidic ones. Because engineered PEDF carries more cationic residues, its pI is significantly higher than that of wild type PEDF. A high pI (>7.4) suggests that modified PEDF is positively charged under physiological conditions (˜pH 7.4) (FIG. 3). See also FIG. 9 for structural references including locations of α-helices and β-strands.

### Example 4: Hyaluronic Acid Binding to Mutated PEDF

### Electrophoresis Studies

The collected medium was concentrated 20× using a centrifuge. Once insoluble substances were removed, the concentrated solution was applied to a column packed with immobilized heparin. The binding process was repeated three times, followed by a washing step to remove the proteins that bound weakly to heparin. Washing employed a phosphate buffer with the concentration of NaCl gradually increasing from 0.05 to 0.5 M. It was observed that 0.15 M NaCl eluted wild type PEDF from immobilized heparin, but engineered PEDF required 0.2-0.5 M NaCl, depending on cationic residue density. After purity and yield were characterized by electrophoresis, PEDF was cryopreserved for long term storage. See also FIG. 3, where PEDF Wild type refers to the protein having SEQ ID NO. 3, and (6) refers to the engineered protein having SEQ ID NO. 11.

Protein electrophoresis followed a standard SDS-PAGE protocol. Briefly, protein samples were mixed with Laemmli sample buffer, boiled to denature, and then loaded onto a 10% polyacrylamide gel. After separations, the proteins were stained with Coomassie blue dye. The molecular weight of engineered PEDF was confirmed using commercial protein standards.

### ELISA Studies

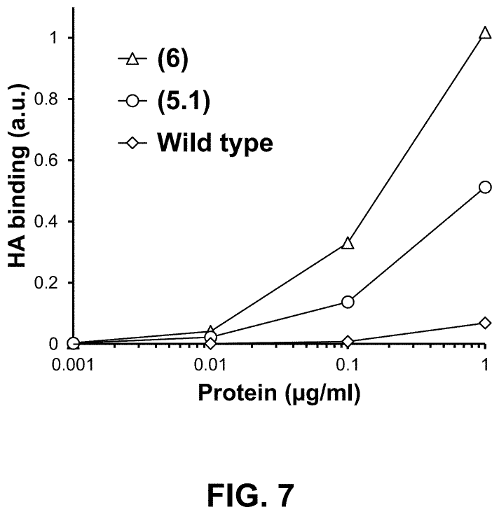

Affinity of individual engineered PEDF proteins for hyaluronic acid was examined using an ELISA assay. Briefly, hyaluronic acid was immobilized on a surface and either wild type (control) or engineered PEDF was allowed to bind. Following washing to remove any non-bound PEDF proteins, an anti-PEDF antibody was used to quantify the amount of PEDF bound to immobilized hyaluronic acid, with a higher value indicating a stronger affinity for hyaluronic acid. See also FIG. 7, where Wild type refers to the protein having SEQ ID NO. 3, (5.1) refers to the engineered protein having SEQ ID NO. 7, and (6) refers to the engineered protein having SEQ ID NO. 11.

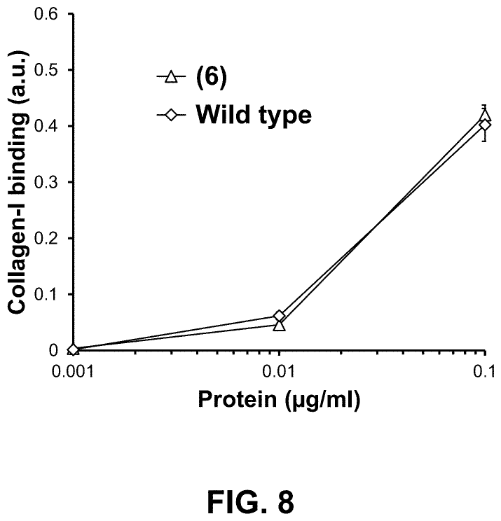

Affinity of individual engineered PEDF proteins for collagen-I was measured using an ELISA assay. Briefly, wild type or engineered PEDF was added to a 96-well plate coated with rat collagen-I and incubated at 4° C. Following washing to remove non-bound PEDF proteins, an anti-PEDF antibody was used to determine the amount of PEDF bound to collagen-I. See also FIG. 8, where Wild type refers to the protein having SEQ ID NO.3, and (6) refers to the engineered protein having SEQ ID NO. 11.

### Example 5: Inhibition of Angiogenesis

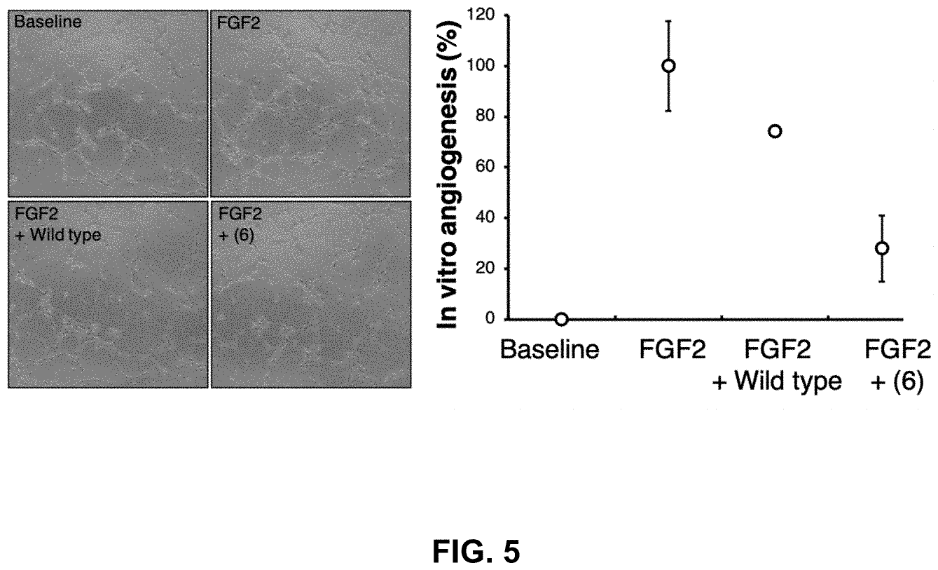

Inhibition of endothelial cell (EC) tube formation was used to determine the bioactivity of engineered PEDF. Briefly, cells from an immortalized human microvascular endothelial cell line (HMEC-1) were maintained in endothelial cell culture medium supplemented with angiogenic growth factors. To form EC tube structure, HMEC-1 cells were seeded in a 96-well plate with Cultrex (R&D Systems). The medium contained 50 ng/mL of fibroblast growth factor 2 (FGF2) to stimulate tube formation as well as different amounts of wild type or engineered PEDF to counteract FGF2 activity. 4 hours after incubation, representative images were recorded and analyzed using ImageJ software (NIH). See also FIG. 5, where Wild type refers to the protein having SEQ ID NO. 3, and (6) refers to the engineered protein having SEQ ID NO. 11.

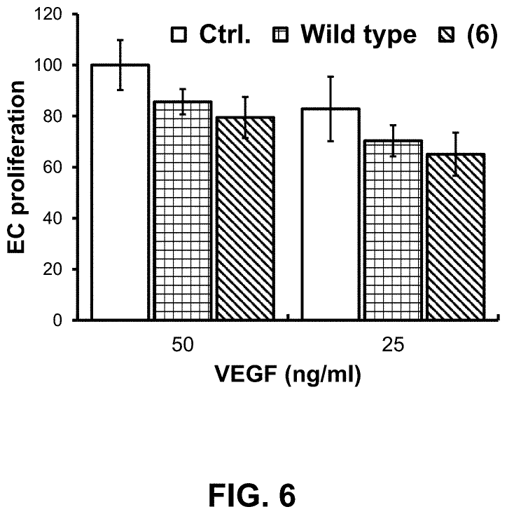

Inhibition of EC proliferation also confirmed that engineered PEDF preserved the bioactivity of wild type PEDF (FIG. 8). Briefly, HMEC-1 cells in the basal medium were seeded for overnight. The medium contained 50 or 25 ng/mL of vascular endothelial growth factor-165 (VEGF) without or with wild type or engineered PEDF was added and incubated for 24 h. After washing with phosphate buffered saline, 0.5% crystal violet staining solution was added to each well and incubate for 20 min. After washing with water, methanol was added, and the absorbance (570) nm was recorded by a plate reader. See also FIG. 6, where Wild type refers to the protein having SEQ ID NO. 3, and (6) refers to the engineered protein having SEQ ID NO. 11.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

What is claimed is:

1. A modified serine proteinase inhibitor comprising anionic amino acid residues and cationic amino acid residues, wherein the sum of the cationic residues is greater than the sum of the anionic residues in the modified serine proteinase inhibitor.

2. The modified serine proteinase inhibitor of claim 1, wherein one or more amino acid residues of the corresponding naturally-occurring serine proteinase inhibitor are replaced with a cationic amino acid residue.

3. The modified serine proteinase inhibitor of claim 1, wherein one or more anionic amino acid residues of the corresponding naturally-occurring serine proteinase inhibitor are replaced with a cationic amino acid residue.

4. The modified serine proteinase inhibitor of claim 3, wherein the one or more anionic amino acid residues replaced with the cationic amino acid residue are present between s2A and hE of the naturally-occurring serine proteinase inhibitor, between hF and s3A of the naturally-occurring serine proteinase inhibitor, or a combination thereof.

5. The modified serine proteinase inhibitor of claim 1, wherein one or more anionic amino acid residues of the corresponding naturally-occurring serine proteinase inhibitor are removed.

6. The modified serine proteinase inhibitor of claim 5, wherein the one or more anionic amino acid residues are present between s2A and hE of the naturally-occurring serine proteinase inhibitor, between hF and s3A of the naturally-occurring serine proteinase inhibitor, or a combination thereof are removed.

7. The modified serine proteinase inhibitor of claim 1, wherein one or more cationic acid residues are added to the corresponding naturally-occurring serine proteinase inhibitor.

8. The modified serine proteinase inhibitor of claim 7, wherein the one or more cationic amino acid residues are added between s2A and hE of the naturally-occurring serine proteinase inhibitor, between hF and s3A of the naturally-occurring serine proteinase inhibitor, or a combination thereof.

9. The modified serine proteinase inhibitor of claim 1, wherein the naturally-occurring serine proteinase inhibitor comprises alpha 1-antitrypsin, C1 esterase Inhibitor, antithrombin, plasminogen activator inhibitor, pigment epithelium-derived factor, or a combination thereof.

10. The modified serine proteinase inhibitor of claim 1, wherein the cationic amino acid residue comprises lysine, arginine, histidine, or any combination thereof.

11. The modified serine proteinase inhibitor of claim 1, wherein the anionic amino acid residue comprises aspartic acid, glutamic acid, or a combination thereof.

12. The modified serine proteinase inhibitor of claim 1, wherein the modified serine proteinase inhibitor has an isoelectric point (pI) that is from 10% to 90% greater than the naturally-occurring serine proteinase inhibitor.

13. The modified serine proteinase inhibitor of claim 1, wherein the sum of the lysine and arginine residues is greater than the sum of the aspartate and glutamate residues in the modified serine proteinase inhibitor.

14. The modified serine proteinase inhibitor of claim 1, wherein the sum of the lysine and arginine residues is from 46 to 55.

15. The modified serine proteinase inhibitor of claim 1, wherein the modified serine proteinase inhibitor is pigment epithelium derived factor (PEDF), and wherein the sum of the cationic amino acid residues present between hF and s3A is greater than the sum of anionic amino acid residues present between hF and s3A.

16. The modified serine proteinase inhibitor of claim 1, wherein the modified serine proteinase inhibitor is pigment epithelium derived factor (PEDF), wherein the sum of the cationic amino acid residues present between s2A and hE is greater than the sum of anionic amino acid residues present between s2A and hE.

17. The modified serine proteinase inhibitor of SEQ ID NO. 5, 7, 9, 11, 13, or a combination thereof.

18. A method for treating a condition relating to increased vascular permeability, a condition relating to increased angiogenesis, an ocular disease, a condition associated with reduced bone mass, skin aging, a wound, a cancer, or a condition associated with inflammation in a subject, the method comprising administering to the subject the modified serine proteinase inhibitor of claim 1.

19. A DNA construct comprising SEQ ID NO. 4, 6, 8, 10, or 12 or at least 70% homology thereto.

20. A vector comprising the DNA construct of claim 19.

21. The vector of claim 20, wherein the vector is a plasmid.

22. The vector of claim 21, wherein the plasmid comprises pWLneo, pSV2cat, pOG44, pXT1, pSG, pSVK3, pBSK, pBSKII, pYES, pYES2, pUC, pUC19, pETDuet-1, p3xFLAG, pBApo, pBI, pcDNA, pCEP, pCI, pCMV, pCTAP, pDEST, pEF, pFLAG, pFN, phCMV, pHet, pIRES, pmRi, pNTAP, prHom, pSecTag, pT-Rex, pTet, pTracer, pTRE, pVAX, pX330, pZeoSV2, a derivative or variant thereof, or a combination thereof.

23. The vector of claim 20, wherein the vector comprises an adenovirus or an adeno-associated virus.

24. A vector comprising RNA, wherein the RNA is complementary to the DNA construct of claim 19.

25. The vector of claim 24, wherein the vector comprises a lentivirus or Sendai virus.

26. The vector of claim 24, wherein the vector comprises mRNA.

27. A biological device comprising host cells transformed with the DNA construct of claim 19.

28. A modified serine proteinase inhibitor produced by culturing the biological device of claim 27.


SEQUENCE LISTING (13 sequences)

SEQ ID NO: 1 - DNA, 1257 bp, Artificial sequence
  Synthetic construct
  misc_feature (291)..(292): n is a, c, g, or t
atgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggannagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaa

SEQ ID NO: 2 - DNA, 1257 bp, Artificial sequence
  Synthetic construct
atgcaggccctggtgctgctgctgtgcattggtgccctgctgggtcatagtagttgtcagaatcccgctagtcctcccgaagaaggctctcccgaccctgatagcaccggcgccctggtggaggaggaggaccctttctttaaggtgccagtgaacaagctggccgctgccgtgtctaatttcggctacgatctgtatcgggtgaggagctccacaagccccaccacaaacgtgctgctgtcccctctgtctgtggctaccgccctgagcgccctgagcctgggagctgagcagagaacagagtccatcatccaccgcgctctgtactatgacctgatctctagccccgatatccacggcacctacaaggagctgctggacaccgtgacagctcctcagaagaacctgaagagcgcctcccggatcgtgttcgagaagaagctgaggatcaagtcctcttttgtggccccactggagaagtcttatggcaccagacctcgcgtgctgacaggcaatccaagactggatctgcaggagatcaacaattgggtgcaggctcagatgaagggcaagctggcccgcagcaccaaggagatccccgacgagatctccatcctgctgctgggcgtggctcacttcaagggccagtgggtgaccaagtttgatagccggaagacatccctggaggacttctacctggatgaggagcggacagtgagggtgcccatgatgtccgaccctaaggctgtgctgagatatggcctggactctgatctgagctgcaagatcgcccagctgcctctgaccggctctatgagcatcatcttctttctgccactgaaggtgacccagaatctgacactgatcgaggagtccctgacatctgagtttatccacgacatcgatcgggagctgaagaccgtgcaggccgtgctgacagtgcctaagctgaagctgtcctacgagggcgaggtgaccaagtctctgcaggagatgaagctgcagtctctgttcgacagcccagatttttccaagatcaccggcaagcccatcaagctgacacaggtggagcacagagctggattcgagtggaacgaggacggagctggaaccacaccaagcccaggcctgcagccagctcacctgacctttcccctggattatcacctgaatcagcccttcatctttgtgctgcgcgacaccgatacaggcgccctgctgtttatcggcaagatcctggaccctcggggaccataa

SEQ ID NO: 3 - protein, 418 aa, Artificial sequence
  Synthetic construct
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 4 - DNA, 1257 bp, Artificial sequence
  Synthetic construct
atgcaggccctggtgctgctgctgtgcattggtgccctgctgggtcatagtagttgtcagaatcccgctagtcctcccgaagaaggctctcccgaccctgatagcaccggcgccctggtggaggaggaggaccctttctttaaggtgccagtgaacaagctggccgctgccgtgtctaatttcggctacgatctgtatcgggtgaggagctccacaagccccaccacaaacgtgctgctgtcccctctgtctgtggctaccgccctgagcgccctgagcctgggagctgagcagagaacagagtccatcatccaccgcgctctgtactatgacctgatctctagccccgatatccacggcacctacaaggagctgctggacaccgtgacagctcctcagaagaacctgaagagcgcctcccggatcgtgttcgagaagaagctgaggatcaagtcctcttttgtggccccactggagaagtcttatggcaccagacctcgcgtgctgacaggcaatccaagactggatctgcaggagatcaacaattgggtgcaggctcagatgaagggcaagctggcccgcagcaccaagaagatccccaagaagatctccatcctgctgctgggcgtggctcacttcaagggccagtgggtgaccaagtttgatagccggaagacatccctggaggacttctacctggatgaggagcggacagtgagggtgcccatgatgtccgaccctaaggctgtgctgagatatggcctggactctgatctgagctgcaagatcgcccagctgcctctgaccggctctatgagcatcatcttctttctgccactgaaggtgacccagaatctgacactgatcgaggagtccctgacatctgagtttatccacgacatcgatcgggagctgaagaccgtgcaggccgtgctgacagtgcctaagctgaagctgtcctacgagggcgaggtgaccaagtctctgcaggagatgaagctgcagtctctgttcgacagcccagatttttccaagatcaccggcaagcccatcaagctgacacaggtggagcacagagctggattcgagtggaacgaggacggagctggaaccacaccaagcccaggcctgcagccagctcacctgacctttcccctggattatcacctgaatcagcccttcatctttgtgctgcgcgacaccgatacaggcgccctgctgtttatcggcaagatcctggaccctcggggaccataa

SEQ ID NO: 5 - protein, 418 aa, Artificial sequence
  Synthetic construct
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKKIPKKISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 6 - DNA, 1257 bp, Artificial sequence
  Synthetic construct
atgcaggccctggtgctgctgctgtgcattggtgccctgctgggtcatagtagttgtcagaatcccgctagtcctcccgaagaaggctctcccgaccctgatagcaccggcgccctggtggaggaggaggaccctttctttaaggtgccagtgaacaagctggccgctgccgtgtctaatttcggctacgatctgtatcgggtgaggagctccacaagccccaccacaaacgtgctgctgtcccctctgtctgtggctaccgccctgagcgccctgagcctgggagctgagcagagaacagagtccatcatccaccgcgctctgtactatgacctgatctctagccccgatatccacggcacctacaaggagctgctggacaccgtgacagctcctcagaagaacctgaagagcgcctcccggatcgtgttcgagaagaagctgaggatcaagtcctcttttgtggccccactggagaagtcttatggcaccagacctcgcgtgctgacaggcaatccaagactggatctgcaggagatcaacaattgggtgcaggctcagatgaagggcaagctgaagcgcagcaagaagaagatccccaagaagatctccatcctgctgctgggcgtggctcacttcaagggccagtgggtgaccaagtttgatagccggaagacatccctggaggacttctacctggatgaggagcggacagtgagggtgcccatgatgtccgaccctaaggctgtgctgagatatggcctggactctgatctgagctgcaagatcgcccagctgcctctgaccggctctatgagcatcatcttctttctgccactgaaggtgacccagaatctgacactgatcgaggagtccctgacatctgagtttatccacgacatcgatcgggagctgaagaccgtgcaggccgtgctgacagtgcctaagctgaagctgtcctacgagggcgaggtgaccaagtctctgcaggagatgaagctgcagtctctgttcgacagcccagatttttccaagatcaccggcaagcccatcaagctgacacaggtggagcacagagctggattcgagtggaacgaggacggagctggaaccacaccaagcccaggcctgcagccagctcacctgacctttcccctggattatcacctgaatcagcccttcatctttgtgctgcgcgacaccgatacaggcgccctgctgtttatcggcaagatcctggaccctcggggaccataa

SEQ ID NO: 7 - protein, 418 aa, Artificial sequence
  Synthetic construct
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLKRSKKKIPKKISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 8 - DNA, 1257 bp, Artificial sequence
  Synthetic construct
atgcaggccctggtgctgctgctgtgcattggtgccctgctgggtcatagtagttgtcagaatcccgctagtcctcccgaagaaggctctcccgaccctgatagcaccggcgccctggtggaggaggaggaccctttctttaaggtgccagtgaacaagctggccgctgccgtgtctaatttcggctacgatctgtatcgggtgaggagctccacaagccccaccacaaacgtgctgctgtcccctctgtctgtggctaccgccctgagcgccctgagcctgggagctgagcagagaacagagtccatcatccaccgcgctctgtactatgacctgatctctagccccgatatccacggcacctacaaggagctgctggacaccgtgacagctcctcagaagaacctgaagagcgcctcccggatcgtgttcgagaagaagctgaggatcaagtcctcttttgtggccccactggagaagtcttatggcaccagacctcgcgtgctgacaaagaatccaagactggatctgcaggagatcaacaattgggtgcagaagcagatgaagggcaagctgaagcgcagcaccaaggagatccccaagcggatctccatcctgctgctgggcgtggctcacttcaagggccagtgggtgaccaagtttgatagccggaagacatccctggaggacttctacctggatgaggagcggacagtgagggtgcccatgatgtccgaccctaaggctgtgctgagatatggcctggactctgatctgagctgcaagatcgcccagctgcctctgaccggctctatgagcatcatcttctttctgccactgaaggtgacccagaatctgacactgatcgaggagtccctgacatctgagtttatccacgacatcgatcgggagctgaagaccgtgcaggccgtgctgacagtgcctaagctgaagctgtcctacgagggcgaggtgaccaagtctctgcaggagatgaagctgcagtctctgttcgacagcccagatttttccaagatcaccggcaagcccatcaagctgacacaggtggagcacagagctggattcgagtggaacgaggacggagctggaaccacaccaagcccaggcctgcagccagctcacctgacctttcccctggattatcacctgaatcagcccttcatctttgtgctgcgcgacaccgatacaggcgccctgctgtttatcggcaagatcctggaccctcggggaccataa

SEQ ID NO: 9 - protein, 418 aa, Artificial sequence
  Synthetic construct
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTKNPRLDLQEINNWVQKQMKGKLKRSTKEIPKRISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 10 - DNA, 1257 bp, Artificial sequence
  Synthetic construct
atgcaggccctggtgctgctgctgtgcattggtgccctgctgggtcatagtagttgtcagaatcccgctagtcctcccgaagaaggctctcccgaccctgatagcaccggcgccctggtggaggaggaggaccctttctttaaggtgccagtgaacaagctggccgctgccgtgtctaatttcggctacgatctgtatcgggtgaggagctccacaagccccaccacaaacgtgctgctgtcccctctgtctgtggctaccgccctgagcgccctgagcctgggagctgagcagagaacagagtccatcatccaccgcgctctgtactatgacctgatctctagccccgatatccacggcacctacaaggagctgctggacaccgtgacagctcctcagaagaacctgaagagcgcctcccggatcgtgttcgagaagaagctgaggatcaagtcctcttttgtggccccactggagaagtcttatggcaccagacctcgcgtgctgacaaagaatccaagactggatctgcaggagatcaacaattgggtgcagaagcagatgaagggcaagctgaagcgcagcaccaagaagatccccaagaagatctccatcctgctgctgggcgtggctcacttcaagggccagtgggtgaccaagtttgatagccggaagacatccctggaggacttctacctggatgaggagcggacagtgagggtgcccatgatgtccgaccctaaggctgtgctgagatatggcctggactctgatctgagctgcaagatcgcccagctgcctctgaccggctctatgagcatcatcttctttctgccactgaaggtgacccagaatctgacactgatcgaggagtccctgacatctgagtttatccacgacatcgatcgggagctgaagaccgtgcaggccgtgctgacagtgcctaagctgaagctgtcctacgagggcgaggtgaccaagtctctgcaggagatgaagctgcagtctctgttcgacagcccagatttttccaagatcaccggcaagcccatcaagctgacacaggtggagcacagagctggattcgagtggaacgaggacggagctggaaccacaccaagcccaggcctgcagccagctcacctgacctttcccctggattatcacctgaatcagcccttcatctttgtgctgcgcgacaccgatacaggcgccctgctgtttatcggcaagatcctggaccctcggggaccataa

SEQ ID NO: 11 - protein, 418 aa, Artificial sequence
  Synthetic construct
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTKNPRLDLQEINNWVQKQMKGKLKRSTKKIPKKISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 12 - DNA, 1257 bp, Artificial sequence
  Synthetic construct
atgcaggccctggtgctgctgctgtgcattggtgccctgctgggtcatagtagttgtcagaatcccgctagtcctcccgaagaaggctctcccgaccctgatagcaccggcgccctggtggaggaggaggaccctttctttaaggtgccagtgaacaagctggccgctgccgtgtctaatttcggctacgatctgtatcgggtgaggagctccacaagccccaccacaaacgtgctgctgtcccctctgtctgtggctaccgccctgagcgccctgagcctgggagctgagcagagaacagagtccatcatccaccgcgctctgtactatgacctgatctctagccccgatatccacggcacctacaaggagctgctggacaccgtgacagctcctcagaagaacctgaagagcgcctcccggatcgtgttcgagaagaagctgaggatcaagtcctcttttgtggccccactgaagaagtcttatggcaccagacctcgcgtgctgacaaagaatccaagactggatctgcaggagatcaacaattgggtgcagaagcagatgaagggcaagctgaagcgcagcaccaagaagatccccaagaagatctccatcctgctgctgggcgtggctcacttcaagggccagtgggtgaccaagtttgatagccggaagacatccctggaggacttctacctggatgaggagcggacagtgagggtgcccatgatgtccgaccctaaggctgtgctgagatatggcctggactctgatctgagctgcaagatcgcccagctgcctctgaccggctctatgagcatcatcttctttctgccactgaaggtgacccagaatctgacactgatcgaggagtccctgacatctgagtttatccacgacatcgatcgggagctgaagaccgtgcaggccgtgctgacagtgcctaagctgaagctgtcctacgagggcgaggtgaccaagtctctgcaggagatgaagctgcagtctctgttcgacagcccagatttttccaagatcaccggcaagcccatcaagctgacacaggtggagcacagagctggattcgagtggaacgaggacggagctggaaccacaccaagcccaggcctgcagccagctcacctgacctttcccctggattatcacctgaatcagcccttcatctttgtgctgcgcgacaccgatacaggcgccctgctgtttatcggcaagatcctggaccctcggggaccataa

SEQ ID NO: 13 - protein, 418 aa, Artificial sequence
  Synthetic construct
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLKKSYGTRPRVLTKNPRLDLQEINNWVQKQMKGKLKRSTKKIPKKISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP